(19) United States
(12) Patent Application Publication
(10) Pub. No.: US 2026/0258451 A1
(43) Pub. Date: Sep. 3, 2026

(54) TRANSIENT CELLULAR REPROGRAMMING FOR REVERSAL OF CELL AGING

(71) Applicant: THE BOARD OF TRUSTEES OF THE LELAND STANFORD JUNIOR UNIVERSITY, Stanford, CA (US)

(72) Inventors: Vittorio SEBASTIANO, Stanford, CA (US); Tapash J. SARKAR, Stanford, CA (US)

(21) Appl. No.: 19/537,394

(22) Filed: Feb. 11, 2026

Related U.S. Application Data
(63) Continuation of application No. 16/979,842, filed on Mar. 13, 2019, now abandoned.
(60) Provisional application No. 62/642,538, filed on Mar. 13, 2018.

(51) Int. Cl. C12N 15/89; A61K 48/00; C12N 5/0775

(52) U.S. Cl. CPC C12N 15/89; A61K 48/0083; C12N 5/0668

(57) ABSTRACT

Provided herein are methods and compositions useful in cellular rejuvenation, tissue engineering, and regenerative medicine. Compositions and methods for rejuvenating aged cells and tissues to restore functionality are disclosed. In particular, cells are rejuvenated by transient exposure to non-integrated mRNAs encoding reprograming factors to rejuvenate cells while retaining cells in a differentiated state.

### CROSS REFERENCE TO RELATED APPLICATIONS

This application is a continuation of U.S. application Ser. No. 16/979,842, filed Sep. 10, 2020, which a U.S. National Stage Application of International Application No. PCT/US2019/022149, filed Mar. 13, 2019, which claims priority benefit to U.S. Provisional Application No. 62/642,538, filed Mar. 13, 2018, the contents of each of which are hereby incorporated by reference in their entireties and for all purposes.

### BACKGROUND

Aging is characterized by a gradual loss of function occurring at the molecular, cellular, tissue and organismal levels. At the chromatin level, aging is associated with the progressive accumulation of epigenetic errors that eventually lead to aberrant gene regulation, stem cell exhaustion, senescence, and deregulated cell/tissue homeostasis. The technology of nuclear reprogramming to pluripotency, through over-expression of a small number of transcription factors, can revert both the age and the identity of any cell to that of an embryonic cell by driving epigenetic reprogramming. The undesirable erasure of cell identity is problematical for the development of rejuvenative therapies because of the resulting destruction of the structure, function and cell type distribution in tissues and organs.

### BRIEF SUMMARY

In view of the foregoing, there is a need for improved methods of rejuvenating cells that avoid dedifferentiation and loss of cell identity. The present disclosure addresses this need, and provides additional benefits as well.

The present disclosure pertains generally to cellular rejuvenation, tissue engineering, and regenerative medicine. In particular, the disclosure relates to compositions and methods for rejuvenating aged cells and tissues to restore functionality by transient exposure to non-integrated mRNAs encoding reprograming factors that rejuvenate cells while retaining cells in a differentiated state.

The disclosure relates to cell-based therapies utilizing rejuvenated cells. In particular, the disclosure relates to methods for rejuvenating aged cells and tissues to restore functionality by transient exposure to non-integrated mRNAs encoding reprograming factors that rejuvenate cells while retaining cells in a differentiated state.

In an aspect, provided herein are methods of rejuvenating cells, the methods including transfecting cells with one or more non-integrative messenger RNAs encoding one or more cellular reprogramming factors for not more than five (5) continuous days, thereby producing rejuvenated cells.

In an aspect, provided herein are method for treating a subject for an age-related disease or condition, a cartilage degeneration disorder, a neurodegenerative disorder, and/or musculoskeletal dysfunction. The methods include administering a therapeutically effective amount of cells that include one or more non-integrative messenger RNAs encoding one or more cellular reprogramming factors.

In an aspect, provided herein are method for treating a subject for an age-related disease or condition, a cartilage degeneration disorder, and/or subject has a musculoskeletal dysfunction. The methods include administering a therapeutically effective amount of one or more non-integrative messenger RNAs encoding one or more cellular reprogramming factors.

In an aspect, provided herein are methods of rejuvenating engineered tissue ex vivo. The methods include transfecting the tissue with one or more non-integrative messenger RNAs encoding one or more cellular reprogramming factors for not more than five (5) continuous days, thereby producing rejuvenated engineered tissue.

In an aspect, provided herein are pharmaceutical compositions including rejuvenated cells obtained by transfecting cells with one or more non-integrative messenger RNAs encoding one or more cellular reprogramming factors for not more than five (5) continuous days.

Thus, in one aspect, the disclosure includes a method of rejuvenating cells, the method comprising: a) transfecting the cells with one or more non-integrative messenger RNAs encoding one or more cellular reprogramming factors, wherein said transfecting is performed once daily for at least two days and not more than 4 days; and b) translating the one or more non-integrative messenger RNAs to produce the one or more cellular reprogramming factors in the cells resulting in transient reprogramming of the cells, wherein the cells are rejuvenated without dedifferentiation into stem cells. The method may be performed on the cells in vitro, ex vivo, or in vivo.

In certain embodiments, transfection with one or more non-integrative messenger RNAs encoding one or more cellular reprogramming factors is performed once daily for 2 days, 3 days, or 4 days.

In certain embodiments, the one or more cellular reprogramming factors are selected from the group consisting of OCT4, SOX2, KLF4, c-MYC, LIN28 and NANOG. In one embodiment, the one or more cellular reprogramming factors comprise OCT4, SOX2, KLF4, c-MYC, LIN28 and NANOG.

The method can be performed on any type of cell. In some embodiments, the cells are mammalian cells (e.g. human, non-human primate, rodent, cat, dog, cow, horse, pig, goat, etc.). For example, the method can be performed on fibroblasts, endothelial cells, chondrocytes, or skeletal muscle stem cells. In another embodiment, the cells are from an elderly subject.

In certain embodiments, the transient reprogramming results in increased expression of HP1γ, H3K9me3, lamina support protein LAP2α, and SIRT1, decreased expression of GMSCF, IL18, and TNFα, decreased nuclear folding, decreased blebbing, increased cell autophagosome formation, increased chymotrypsin-like proteasome activity, increased mitochondria membrane potential, or decreased reactive oxygen species (ROS).

In certain embodiments, the cells are within a tissue or organ. Transient reprogramming, according to the methods described herein, may restore function of the cells in the tissue or organ, increase potency of cells in the tissue or organ, reduce the numbers of senescent cells within the tissue or organ, enhance replicative capacity of cells within the tissue or organ, or extend the life span of cells within the tissue or organ.

In another aspect, the disclosure includes a method for treating a subject for an age-related disease or condition, the method comprising: a) transfecting cells of the subject with one or more non-integrative messenger RNAs encoding one or more cellular reprogramming factors, wherein said transfecting is performed once daily for at least two days and not more than 4 days; and b) expressing the one or more cellular reprogramming factors in the cells in the subject resulting in transient reprogramming of the cells, wherein the cells are rejuvenated without dedifferentiation into stem cells. The cells may be transfected, ex vivo or in vivo.

In certain embodiments, the one or more cellular reprogramming factors are selected from the group consisting of OCT4, SOX2, KLF4, c-MYC, LIN28 and NANOG. In one embodiment, the one or more cellular reprogramming factors comprise OCT4, SOX2, KLF4, c-MYC, LIN28 and NANOG.

In certain embodiments, the age-related disease or condition is a degenerative disease, a neurodegenerative disease, a cardiovascular disease, a peripheral vascular disease, a dermatologic disease, an eye disease, an autoimmune disease, an endocrine disorder, a metabolic disorder, a musculoskeletal disorder, a disease of the digestive system, or a respiratory disease.

In another embodiment, the disclosure includes a method for treating a subject for a disease or disorder involving cartilage degeneration, the method comprising: a) transfecting chondrocytes of the subject with one or more non-integrative messenger RNAs encoding one or more cellular reprogramming factors, wherein said transfecting is performed once daily for at least two days and not more than 4 days; and b) expressing the one or more cellular reprogramming factors in the chondrocytes resulting in transient reprogramming of the chondrocytes, wherein the chondrocytes are rejuvenated without dedifferentiation into stem cells. The rejuvenated chondrocytes may be transplanted, for example, into an arthritic joint of the subject.

The method may be performed ex vivo, in vitro or in vivo. In one embodiment, chondrocytes are isolated from a cartilage sample obtained from the subject and transfected ex vivo, then transplanted into the subject.

In certain embodiments, the disease or disorder involving cartilage degeneration is arthritis (e.g., osteoarthritis or rheumatoid arthritis).

In certain embodiments, treatment reduces inflammation in the subject.

In certain embodiments, treatment reduces expression of RANKL, iNOS, IL6, IL8, BDNF, IFNα, IFNγ, and LIF and increases expression of COL2A1 by the chondrocytes.

In another aspect, the disclosure includes a method for treating a disease or disorder involving muscle degeneration in a subject, the method comprising: a) transfecting skeletal muscle stem cells of the subject with one or more non-integrative messenger RNAs encoding one or more cellular reprogramming factors, wherein said transfecting is performed once daily for at least two days and not more than 4 days; and b) expressing the one or more cellular reprogramming factors in the skeletal muscle stem cells resulting in transient reprogramming of the skeletal muscle stem cells, wherein the skeletal muscle stem cells are rejuvenated without loss of their ability to differentiate into muscle cells.

The method may be performed ex vivo, in vitro or in vivo. In one embodiment, skeletal muscle stem cells are isolated from a muscle tissue sample obtained from the subject and transfected ex vivo, then transplanted into a muscle in need of repair or regeneration in the subject.

In certain embodiments, the one or more cellular reprogramming factors are selected from the group consisting of OCT4, SOX2, KLF4, c-MYC, LIN28 and NANOG. In one embodiment, the one or more cellular reprogramming factors comprise OCT4, SOX2, KLF4, c-MYC, LIN28 and NANOG.

In certain embodiments, treatment restores potency of the skeletal muscle stem cells. In certain embodiments, treatment results in regeneration of myofibers.

The methods of the disclosure may be performed on any subject. In certain embodiments, the subject is a mammal, for example, a human, a non-human primate, a rodent, a cat, a dog, a cow, a horse, a pig, or a goat. In some embodiments, the subject is elderly.

These and other embodiments of the subject disclosure will readily occur to those of skill in the art in view of the disclosure herein.

### BRIEF DESCRIPTION OF THE DRAWINGS

FIGS. 1A-1L show that transient reprogramming reverts aged physiology towards a more youthful state in fibroblasts. FIG. 1A shows a representative plot demonstrating variance in effect (ROS) with duration of treatment: two days of transient reprogramming versus four days, both with two days of subsequent relaxation. All box and bar plots are generated after combining data over all individual cells, biological and technical replicates for ease of viewing. Significance level shown allows a grace on one pairwise comparison, for patient to patient variability. FIG. 1B shows quantification of single cell levels of heterochromatin markers H3K9me3 and HP1γ using immunocytochemistry. FIG. 1C shows quantification of presence of nuclear laminar support polypeptide LAP2α in single cells and percent of abnormal nuclei (folded or blebbed) in each population using immunocytochemistry. FIG. 1D shows results of live cells imaging with florescent-tagged substrates cleaved during autophagosome formation in single cells and chymotrypsin like 20S proteolytic activity in total population. FIG. 1E shows individual cell mitochondrial membrane potential and ROS levels quantified with mitochondria specific dyes. FIG. 1F shows single cell quantification of immunostaining for SIRT1. FIG. 1G shows results from telomere quantitative fluorescent in situ hybridization (QFISH) on single cell. FIG. 1H shows results from SAβGal staining for senescent populations. FIG. 1I shows quantification of inflammatory cytokine profiling using panels of analyte antibody conjugated beads for multiplex cytometry. FIG. 1J shows representative plot showing maintenance of youthful shifts for longer periods of relaxation, 4 and 6 days after 4 day transient reprogramming. Cells in each cohort were then subjected to 80 base pair paired end read RNA sequencing to yield transcriptomic profiles for each group (Young, Aged and Treated—R4X2). FIG. 1K shows Principal Components Analysis in the subspace defined by the aging signature. Arrows clarify which data points are Treated, Aged, or Young. FIG. 1L shows a comparison of log fold change between young and aged (x-axis) and treated and aged (y-axis). Dark grey points are all the genes of the aging signature while light grey points are the genes that also overlap with the treatment signature, significantly differ between treated and aged. A majority of the genes lie along y=x line, signifying that the magnitude of changes by treatment closely matched the magnitude of difference between Young and Aged. Significance is calculated with students t-test, pairwise between treated and aged, and group wise when comparing to young patients. P value: *<0.05, **<0.01, ***<0.001, dark-filled asterisks indicate comparison to Young and light-filled asterisks indicate comparison to Aged.

FIGS. 2A-2K show that transient reprogramming reverts aged physiology towards a more youthful state in Endothelial Cells. FIG. 2A shows a representative plot demonstrating variance in effect (ROS) with duration of treatment: two days of transient reprogramming versus four days, both with two days of subsequent relaxation. All box and bar plots are generated after combining data over all individual cells, biological and technical replicates for ease of viewing. Significance level shown allows a grace on one pairwise comparison, for patient to patient variability. FIG. 2B shows quantification of single cell levels of heterochromatin markers, H3K9me3 and HP1γ, using immunocytochemistry. FIG. 2C shows quantification of presence of nuclear laminar support polypeptide LAP2α in single cells and percent of abnormal nuclei (folded or blebbed) in each population using immunocytochemistry. FIG. 2D shows results of live cells imaging with florescent tagged substrates cleaved during autophagosome formation in single cells and chymotrypsin like 20S proteolytic activity in total population. FIG. 2E shows individual cell mitochondria membrane potential and ROS levels quantified with mitochondria specific dyes. FIG. 2F shows single cell quantification of immunostaining for SIRT1. FIG. 2G shows results from telomere quantitative fluorescent in situ hybridization (QFISH) on single cell. FIG. 2H shows results from SAβGal staining for senescent populations. FIG. 2I shows representative plot showing maintenance of youthful shifts for longer periods of relaxation, 4 and 6 days after 4 day transient reprogramming. Cells in each cohort were then subjected to 80 base pair paired end read RNA sequencing to yield transcriptomic profiles for each group (Young, Aged and Treated—R4X2). FIG. 2J shows Principal Components analysis in the subspace defined by the aging signature. Arrows clarify which data points are Treated, Aged, or Young. FIG. 2K shows a comparison of log fold change between young and aged (x-axis) and treated and aged (y-axis). Dark grey points are all the genes of the aging signature while light grey points are the genes that also overlap with the treatment signature, significantly differ between treated and aged. A majority of the genes lie along y=x line, signifying that the magnitude of changes by treatment closely matched the magnitude of difference between Young and Aged. Significance is calculated with students t-test, pairwise between treated and aged, and group wise when comparing to young patients. P value: *<0.05, **<0.01, *<0.001 dark-filled asterisks indicate comparison to Young and light-filled asterisks indicate comparison to Aged.

FIGS. 3A-3I show that transient reprogramming mitigates osteoarthritis phenotypes in diseased chondrocytes: All box and bar plots are combined over biological and technical replicates for ease of viewing. Significance level shown allows a grace on one pairwise comparison, for patient to patient variability. Treated refers to an optimized three days reprogramming and two days relaxation. FIG. 3A shows population results from cell viability staining. FIG. 3B shows qRT-PCR evaluation of RNA levels of anabolic factors COL2A1. FIG. 3C shows quantification of ATP concentration in each cohort. FIG. 3D shows qRT-PCR evaluation of RNA levels of antioxidant SOD2, note young levels are below OA as SOD2 elevation only benefits when ROS is present, i.e., the OA state (FIG. 3E). FIGS. 3F and 3G show qRT-PCR evaluation of RNA levels of catabolic factors MMP13 (FIG. 3F) and MMP3 (FIG. 3G). FIGS. 3H and 3I show RT-PCR evaluation of RNA levels of pro-inflammatory factors RANKL (FIG. 3H) and iNOS (FIG. 3I) profiling using panels of analyte antibody conjugated beads for multiplex cytometric analysis. Significance is calculated with students t-test, pairwise between treated and aged, and group wise when comparing to young patients P value: *<0.05, **<0.01, ***<0.001 dark-filled asterisks indicate comparison to Young and light-filled asterisks indicate comparison to Aged.

FIGS. 4A-4G show that transient reprogramming restores aged muscle stem cell potency. FIG. 4A shows measurements of MuSC activation from quiescence. Freshly isolated aged MuSCs were incubated with EdU fixed after two days of treatment and one or two days of relaxation. All box and bar plots are combined over biological and technical replicates for ease of viewing. Significance level shown allows a grace on one pairwise comparison, for patient to patient variability. FIG. 4B shows quantified results of bioluminescence, measured from mice 11 days after transplantation in TA muscles of treated/untreated+Luciferase mouse MuSCs, at different time points following transplantation and injury. FIG. 4C shows quantification of immunofluorescence staining of GFP expression in TA muscle cross-sections of mice imaged and quantified in FIG. 4B. FIG. 4D shows quantification of cross sectional area of donor derived GFP+ fibers in TA muscles that were recipients of transplanted MuSCs. FIG. 4E shows results of bioluminescence imaging of TA muscle reinjured after 60 d (second injury) after the transplantation. The second injury was performed to test whether the bioluminescence signal increased as a consequence of activating and expanding luciferase+/GFP+ MuSCs that were initially transplanted and that had engrafted under the basal lamina. FIG. 4F shows quantified results of bioluminescence measured from mice 11 days after transplantation in TA muscles of treated Luciferase+human MuSCs. FIG. 4G shows variation in ratio of bioluminescence between treated and untreated MuSCs obtained from healthy donors of different age groups. Significance is calculated with students t-test, pairwise between treated and aged, and group wise when comparing to young patients. P value: *<0.05, **<0.01, ***<0.001, dark-filled asterisks indicate comparison to Young and light-filled asterisks indicate comparison to Aged.

FIGS. 5A-5J show that transient reprogramming reverts aged physiology towards a more youthful state in human fibroblasts and endothelial cells. Fibroblasts and endothelial cells were obtained from otherwise healthy young and aged individuals. FIG. 5A shows distribution of epigenetic and nuclear markers for H3K9me3. FIG. 5B shows distribution of epigenetic and nuclear markers for HP1γ. FIG. 5C shows distribution of epigenetic and nuclear markers for LAP2α. FIG. 5D shows distribution of nutrients and energy regulation for SIRT1. FIG. 5E shows distribution of nutrients and energy regulation for Mito membrane potential. FIG. 5F shows distribution of nutrients and energy regulation for Mito ROS. FIG. 5G shows distribution and bulk waste clearance and senescence in the autophagosome. FIG. 5H shows proteasomal activity for young, aged, and treated cells. FIG. 5I shows senescence activity for young, aged, and treated cells. FIG. 5J shows secreted cytokines in young, aged, and treated cells.

FIGS. 6A-6I show transcriptomic and methylomic analyses for aged fibroblasts and endothelial cells. FIG. 6A shows the young versus aged transcriptomic profile for fibroblasts. The data shows that 961 genes (5.85%) in fibroblasts (678 upregulated, 289 downregulated) differed between young and aged cells, with the significance criteria of p<05 and a log fold change cutoff +/−0.5. FIG. 6B shows PCA analysis for fibroblasts. FIG. 6C shows expression analysis for fibroblasts. Dark grey points are all the genes of the aging signature while light grey points are the genes that also overlap with the treatment signature, significantly differ between treated and aged. FIG. 6D shows the young versus aged profile for endothelial cells. The data shows 748 genes (4.80%) in endothelial cells (389 upregulated, 377 down regulated) differed between young and aged cells, with the significance criteria of p<0.05 and a log fold change cutoff +/−0.5. FIG. 6E shows PCA analysis for endothelial cells. FIG. 6F shows expression analysis for endothelial cells. Dark grey points are all the genes of the aging signature while light grey points are the genes that also overlap with the treatment signature, significantly differ between treated and aged. FIG. 6G is a graph of methylation age for fibroblasts evaluated by Horvath Clock before and after treatment and the data shows general trend of reduction. FIG. 6H is a graph of methylation age for endothelial cells evaluated by Horvath Clock before and after treatment and the data shows general trend of reduction. FIG. 6I is a dendogram showing unsupervised clustering in methylation patterns separating by treatment status, by sex, by patient and by cell type. Clustering demonstrates a collective retention of cell identity, at least when comparing fibroblasts and endothelial cells.

FIGS. 7A-7M show transient reprogramming in osteoarthritic chondrocytes and mesenchymal stem cells. FIGS. 7A-I are data showing transient reprogramming mitigates inflammatory phenotypes in diseased chondrocytes. Chondrocytes were obtained from aged diagnosed late stage Osteoarthritis (OA) patients from cartilage biopsies. Aged OA cells and transiently reprogrammed OA cells were evaluated for OA specific phenotypes. All box and bar plots are combined over biological and technical replicates for ease of viewing. Overall significance ranking set by second most stringent p value. Significance was calculated with students t-tests P value: *<0.05, **<0.01, ***<0.001. Error bars show RMSE (root mean square error). FIG. 7A shows elevation of ATP levels with treatment in chondrocytes by measurement of ATP concentration using glycerol based fluorophore. FIG. 7B shows ROS activity by following live single cell image of cells up taking superoxide triggered fluorescent dyes shows diminished signal after treatment. FIG. 7C shows results of qRT-PCR evaluation of RNA levels of antioxidant SOD2 which were elevated with treatment. FIG. 7D shows cell proliferation in young, aged and aged-treated chondrocytes with the aged-treated cells shifting towards levels close to young cells. FIG. 7E shows data from qRT-PCR reflecting elevation of RNA levels for extracellular matrix protein component COL2A1 in young, aged and aged-treated chondrocytes with the aged-treated cells shifting towards levels close to young cells. FIG. 7F is data showing qRT-PCR levels of chondrogenic identity and function transcription factor SOX9 is retained after treatment. FIG. 7G summarizes RT-PCR evaluation showing treatment diminishes intracellular RNA levels of the NF-κB ligand RANKL. FIG. 7H summarizes RT-PCR evaluation showing treatment drops levels of iNOS for producing nitric oxide as a response and to propagate inflammatory stimulus with a shift closer to that of young chondrocytes. FIG. 7I is data reflecting cytokine profiling of chondrocyte secretions shows an increase pro-inflammatory cytokines that diminishes with treatment. FIG. 7J is a graph showing patient by patient distribution shift towards reduced levels of p16 with treatment in mesenchymal stem cells. FIG. 7K is a graph showing patient by patent distribution shift towards reduced levels of p21 with treatment in mesenchymal stem cells. FIG. 7L shows fold change corresponding to increase in cell proliferation in aged and treated mesenchymal stem cells. FIG. 7M shows percentage of senescent aged and treated mesenchymal stem cells corresponding to the decrease in cell senescence.

FIGS. 8A-8J show the effects of transient reprogramming of engineered skin tissue. FIGS. 8A-8C show skin senescence parameters for fibroblasts and keratinocytes. FIG. 8A shows histology score, incorporating metrics for morphology, structure and organization, show improvement with mRNA treatment but not with commonly marketed skin treatment, retinoic acid. Reduction in senescence parameters are shown in FIG. 8B (SaβGal) and FIG. 8C left panel (p16) and inflammatory parameters are shown in FIG. 8C center panel (IL-8) and FIG. 8C right panel (MMP-1) with mRNA treatment and further comparison to effects of retinoic acid. FIGS. 8D-8J show muscle regeneration in satellite cells. FIG. 8D shows quantified results of bioluminescence measured from mice 11 days after transplantation in TA muscles of treated Luciferase⁺ human MuSCs. FIG. 8E shows bioluminescence of cohorts aged 10-30 days, aged 30-55 days, and aged 60-80 days. Variation in ratio of bioluminescence between treated and untreated MuSCs obtained from healthy donors of different age groups. Significance is calculated with students t-test, pairwise between treated and aged, and group wise when comparing to young patients (Age groups. 10-30: n=5; 30-55: n=7; 60-80: n=5). P value: *<0.05, *<0.01, *<0.001, dark-filled asterisks indicate comparison to Young and light-filled asterisks indicate comparison to Aged. FIG. 8F shows tetanic force measurements of aged muscles injured and transplanted with aged MuSCs. TA muscles were dissected and electrophysiology ex vivo for tetanic measurement performed. Baseline of force production of untransplanted muscles was measured in young (4 months, blue broken line) and aged (27 months, red broken line) mice. Treated aged MuSCs were transplanted into TA muscles of aged mice and force production measured 30 days later (n=5). FIG. 8G shows quantified results of bioluminescence in of treated, aged, and young cells at different time points following transplantation and injury (n=10). FIG. 8H shows quantification of immunofluorescence staining in TA muscle cross-sections of mice transplanted with aged treated and aged untreated cells (n=5). FIG. 8I is a graph showing quantification of cross sectional area of donor derived GFP+ fibers in TA muscles that were recipients of transplanted MuSCs (n=5). FIG. 8J shows results of bioluminescence imaging of TA muscles reinjured after 60 days (second injury) after MuSC transplantations (n=6). The second injury was performed to test whether the bioluminescence signal increased as a consequence of activating and expanding luciferase⁺/GFP⁺ MuSCs that were initially transplanted and that had engrafted under the basal lamina.

FIGS. 9A-9D show transfection of corneal epithelial cells with transiently reprogrammed cells. FIG. 9A shows reduction in senescence as measured by expression of p16 in aged versus treated cells. FIG. 9B shows reduction in the senescence as measured by expression of p21 in aged versus treated cells. FIG. 9C shows reduction in inflammatory factor IL8 in aged versus treated cells. FIG. 9D shows increase in mitochondria biogenesis as measured by PGC1α expression.

FIGS. 10A-10C are charts showing P-value of change in cell specific markers between treated and aged cells using RNAseq analysis. Out of the 8 Fibroblast and 50 Endothelial Cell markers none showed significant change with treatment in their respective cell types, suggesting retention cell identity.

FIG. 11 shows hallmarks of aging, which were analyzed using a panel of 11 established assays.

### DETAILED DESCRIPTION

The practice of the technology described herein will employ, unless otherwise indicated, conventional methods of medicine, cell biology, pharmacology, chemistry, biochemistry, molecular biology and recombinant DNA techniques, and immunology, within the skill of the art. Such techniques are explained fully in the literature. See, e.g., G. Vunjak-Novakovic and R. I. Freshney Culture of Cells for Tissue Engineering (Wiley-Liss, 1st edition, 2006); Arthritis Research: Methods and Protocols, Vols. 1 and 2: (Methods in Molecular Medicine, Cope ed., Humana Press, 2007); Cartilage and Osteoarthritis (Methods in Molecular Medicine, M. Sabatini P. Pastoureau, and F. De Ceuninck eds., Humana Press; 2004); Handbook of Experimental Immunology, Vols. I-IV (D. M. Weir and C. C. Blackwell eds., Blackwell Scientific Publications); A. L. Lehninger, Biochemistry (Worth Publishers, Inc., current addition); and Sambrook et al., Molecular Cloning: A Laboratory Manual (3rd Edition, 2001).

All publications, patents and patent applications cited herein, whether supra or infra, are hereby incorporated by reference in their entireties.

### I. Definitions

In describing the present disclosure, the following terms will be employed, and are intended to be defined as indicated below.

It must be noted that, as used in this specification and the appended claims, the singular forms “a,” “an” and “the” include plural referents unless the content clearly dictates otherwise. Thus, for example, reference to “a cell” includes a mixture of two or more cells, and the like.

Reference throughout this specification to, for example, “one embodiment”, “an embodiment”, “another embodiment”, “a particular embodiment”, “a related embodiment”, “a certain embodiment”, “an additional embodiment”, or “a further embodiment” or combinations thereof means that a particular feature, structure or characteristic described in connection with the embodiment is included in at least one embodiment of the present disclosure. Thus, the appearances of the foregoing phrases in various places throughout this specification are not necessarily all referring to the same embodiment. Furthermore, the particular features, structures, or characteristics may be combined in any suitable manner in one or more embodiments.

As used herein, the term “about” means a range of values including the specified value, which a person of ordinary skill in the art would consider reasonably similar to the specified value. In embodiments, the term “about” means within a standard deviation using measurements generally acceptable in the art. In embodiments, about means a range extending to +/−10% of the specified value. In embodiments, about means the specified value.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements.

As used herein, the term “biocompatible” generally refers to a material and any metabolites or degradation products thereof that are generally non-toxic to the recipient and do not cause any significant adverse effects to the subject.

As used herein, the term “cell” refers to an intact live cell, naturally occurring or modified. The cell may be isolated from other cells, mixed with other cells in a culture, or within a tissue (partial or intact), or an organism. The methods described herein can be performed, for example, on a sample comprising a single cell, a population of cells, or a tissue or organ comprising cells.

As used herein, the term “non-integrative” with reference to a messenger RNA (mRNA) refers to an mRNA molecule that is not integrated intrachromosomally or extrachromosomally into the host genome, nor integrated into a vector.

As used herein, the term “transfection” refers to the uptake of exogenous DNA or RNA by a cell. A cell has been “transfected” when exogenous DNA or RNA has been introduced inside the cell membrane. A number of transfection techniques are generally known in the art. See, e.g., Graham et al. (1973) Virology, 52:456, Sambrook et al. (2001) Molecular Cloning, a laboratory manual, 3rd edition, Cold Spring Harbor Laboratories, New York, Davis et al. (1995) Basic Methods in Molecular Biology, 2^{nd} edition, McGraw-Hill, and Chu et al. (1981) Gene 13:197. Such techniques can be used to introduce one or more exogenous DNA or RNA molecules into cells. The term refers to both stable and transient uptake of the DNA or RNA molecules. For example, transfection can be used for transient uptake of mRNAs encoding cellular reprogramming factors into cells in need of rejuvenation.

As used herein, the term “transient reprogramming” refers to exposure of cells to cellular reprogramming factors for a period of time sufficient to rejuvenate cells (i.e., eliminate all or some hallmarks of aging), but not long enough to cause dedifferentiation into stem cells. Such transient reprogramming results in rejuvenated cells that retain their identity (i.e., differentiated cell-type).

As used herein, the term “rejuvenated cell(s)” refers to aged cells that have been treated or transiently reprogrammed with one or more cellular reprogramming factors such that the cells have a transcriptomic profile of a younger cell while still retaining one or more cell identity markers.

As used herein, the term “mammalian cell” refers to any cell derived from a mammalian subject suitable for transplantation into the same or a different subject. The cell may be xenogeneic, autologous, or allogeneic. The cell can be a primary cell obtained directly from a mammalian subject. The cell may also be a cell derived from the culture and expansion of a cell obtained from a subject. In some embodiments, the cell has been genetically engineered to express a recombinant protein and/or nucleic acid.

As used herein, the term “stem cell” refers to a cell that retains the ability to renew itself through mitotic cell division and that can differentiate into a diverse range of specialized cell types. Mammalian stem cells can be divided into three broad categories: embryonic stem cells, which are derived from blastocysts, adult stem cells, which are found in adult tissues, and cord blood stem cells, which are found in the umbilical cord. In a developing embryo, stem cells can differentiate into all of the specialized embryonic tissues. In adult organisms, stem cells and progenitor cells act as a repair system for the body by replenishing specialized cells. Totipotent stem cells are produced from the fusion of an egg and sperm cell. Cells produced by the first few divisions of the fertilized egg are also totipotent. These cells can differentiate into embryonic and extraembryonic cell types. Pluripotent stem cells are the descendants of totipotent cells and can differentiate into cells derived from any of the three germ layers. Multipotent stem cells can produce only cells of a closely related family of cells (e.g., hematopoietic stem cells differentiate into red blood cells, white blood cells, platelets, etc.). Unipotent cells can produce only one cell type, but have the property of self-renewal, which distinguishes them from non-stem cells. Induced pluripotent stem cells are a type of pluripotent stem cell derived from adult cells that have been reprogrammed into an embryonic-like pluripotent state. Induced pluripotent stem cells can be derived, for example, from adult somatic cells such as skin or blood cells.

As used herein, the term “transcriptomic profile” refers to the set of all RNA molecules in one cell or a population of cells. It is sometimes used to refer to all RNAs, or just mRNA, depending on the particular experiment. It differs from the exome in that it includes only those RNA molecules found in a specified cell population, and usually includes the amount or concentration of each RNA molecule in addition to the molecular identities. Methods of obtaining a transcriptomic profile include DNA microarrays and next-generation sequencing technologies such as RNA-Seq. Transcription can also be studied at the level of individual cells by single-cell transcriptomics. There are two general methods of inferring transcriptome sequences. One approach maps sequence reads onto a reference genome, either of the organism itself (whose transcriptome is being studied) or of a closely related species. The other approach, de novo transcriptome assembly, uses software to infer transcripts directly from short sequence reads.

As used herein, the term “Root Mean Square Error” or “RMSE” refers to the standard deviation of the residuals (prediction errors). Residuals are a measure of how far from the regression line data points are. RMSE is a measure of how spread out these residuals are. In other words, it tells you how concentrated the data is around the line of best fit.

As used herein, the term “cell viability” refers to a measure of the number of cells that are living or dead, based on a total cell sample. High cell viability, as defined herein, refers to a cell population in which greater than 85% of all cells are viable, preferably greater than 90-95%, and more preferably a population characterized by high cell viability containing more than 99% viable cells.

As used herein, the term “autophagosome” refers to a spherical structure with double layer membranes. It is a key structure in macroautophagy, the intracellular degradation system for cytoplasmic contents (e.g., abnormal intracellular proteins, excess or damaged organelles) and also for invading microorganisms. After formation, autophagosomes deliver cytoplasmic components to the lysosomes. The outer membrane of an autophagosome fuses with a lysosome to form an autolysosome. The lysosome's hydrolases degrade the autophagosome-delivered contents and its inner membrane.

As used herein, the term “proteasome activity” refers to the degradation of unneeded or damaged proteins by the proteasome, a protein complex, through proteolysis, a chemical reaction that breaks peptide bonds. The term “chymotrypsin-like proteasome activity” refers to a distinct catalytic activity of the proteasome.

As used herein, the term “mitochondria membrane potential” refers to the electrical potential and proton gradient that results from redox transformations associated with the activity of the Krebs cycle and serves as an intermediate form of energy storage to make ATP. It is generated by proton pumps and is an essential process of energy storage during oxidative phosphorylation. It plays a key role in mitochondrial homeostasis through selective elimination of dysfunctional mitochondria.

As used herein, the term “pharmaceutically acceptable excipient or carrier” refers to an excipient that may optionally be included in the compositions of the disclosure and that causes no significant adverse toxicological effects to the patient.

As used herein, the term “reactive oxygen species” or “ROS” are chemically reactive chemical species containing oxygen. Examples include peroxides, superoxide, hydroxyl radical, singlet oxygen, and alpha-oxygen. In a biological context, ROS are formed as a natural byproduct of the normal metabolism of oxygen and have important roles in cell signaling and homeostasis.

As used herein, the term “senescence-associated secretory phenotype” or “SASP” refers to an array of diverse cytokines, chemokines, growth factors, and proteases that are a characteristic feature of senescent cells. Senescent cells are stable, non-dividing cells that are still metabolically active and exhibit the upregulation of a wide range of genes including those that encode secreted proteins, such as inflammatory cytokines, chemokines, extracellular matrix remodeling factors, and growth factors. These secreted proteins function physiologically in the tissue microenvironment, in which they could propagate the stress response and communicate with neighboring cells. This phenotype, termed the senescence-associated secretory phenotype (SASP) uncovers the paracrine function of senescent cells, and is an important characteristic that distinguishes senescent cells from non-senescent, cell cycle-arrested cells, such as quiescent cells and terminally differentiated cells. “SASP cytokines” refers specifically to cytokines produced by senescent cells to create the senescence-associated secretory phenotype. The cytokines include but are not limited to IL18, IL1A, GROA, IL22, and IL9.

As used herein, the term “methylation landscape” refers to the DNA methylation pattern of a cell or cell population.

As used herein, the term “epigenetic clock” refers to a biochemical test that can be used to measure age. The test is based on DNA methylation levels. The first multi-tissue epigenetic clock, Horvath's epigenetic clock, or the “Horvath clock” was developed by Steve Horvath (Horvath 2013).

As used herein, the term “cellular reprogramming factors” refers to a set of transcription factors that can convert adult or differentiated cells into pluripotent stem cells. In embodiments herein, the factors include OCT4, SOX2, KLF4, c-MYC, LIN28 and NANOG.

“Pharmaceutically acceptable salt” includes, but is not limited to, amino acid salts, salts prepared with inorganic acids, such as chloride, sulfate, phosphate, diphosphate, bromide, and nitrate salts, or salts prepared from the corresponding inorganic acid form of any of the preceding, e.g., hydrochloride, etc., or salts prepared with an organic acid, such as malate, maleate, fumarate, tartrate, succinate, ethylsuccinate, citrate, acetate, lactate, methanesulfonate, benzoate, ascorbate, para-toluenesulfonate, palmoate, salicylate and stearate, as well as estolate, gluceptate and lactobionate salts. Similarly, salts containing pharmaceutically acceptable cations include, but are not limited to, sodium, potassium, calcium, aluminum, lithium, and ammonium (including substituted ammonium).

As used herein, the term “transplant” refers to the transfer of a cell, tissue, or organ to a subject from another source. The term is not limited to a particular mode of transfer. Cells may be transplanted by any suitable method, such as by injection or surgical implantation.

As used herein, the term “arthritis” includes, but is not limited to, osteoarthritis, rheumatoid arthritis, lupus-associated arthritis, juvenile idiopathic arthritis, reactive arthritis, enteropathic arthritis and psoriatic arthritis.

As used herein, the term “age-related disease or condition” refers to any condition, disease, or disorder associated with aging such as, but not limited to, neurodegenerative diseases (e.g., Alzheimer's disease, Parkinson's disease, Huntington's disease, amyotrophic lateral sclerosis, dementia, and stroke), cardiovascular and peripheral vascular diseases (e.g., atherosclerosis, peripheral arterial disease (PAD), hematomas, calcification, thrombosis, embolisms, and aneurysms), eye diseases (e.g., age-related macular degeneration, glaucoma, cataracts, dry eye, diabetic retinopathy, vision loss), dermatologic diseases (dermal atrophy and thinning, elastolysis and skin wrinkling, sebaceous gland hyperplasia or hypoplasia, senile lentigo and other pigmentation abnormalities, graying hair, hair loss or thinning, and chronic skin ulcers), autoimmune diseases (e.g., polymyalgia rheumatica (PMR), giant cell arteritis (GCA), rheumatoid arthritis (RA), crystal arthropathies, and spondyloarthropathy (SPA)), endocrine and metabolic dysfunction (e.g., adult hypopituitarism, hypothyroidism, apathetic thyrotoxicosis, osteoporosis, diabetes mellitus, adrenal insufficiency, various forms of hypogonadism, and endocrine malignancies), musculoskeletal disorders (e.g., arthritis, osteoporosis, myeloma, gout, Paget's disease, bone fractures, bone marrow failure syndrome, ankylosis, diffuse idiopathic skeletal hyperostosis, hematogenous osteomyelitis, muscle atrophy, peripheral neuropathy, multiple sclerosis, amyotrophic lateral sclerosis (ALS), Duchenne muscular dystrophy, primary lateral sclerosis, and myasthenia gravis), diseases of the digestive system (e.g., liver cirrhosis, liver fibrosis, Barrett's esophagus), respiratory diseases (e.g., pulmonary fibrosis, chronic obstructive pulmonary disease, asthma, chronic bronchitis, pulmonary embolism (PE), lung cancer, and infections), and any other diseases and disorders associated with aging.

As used herein, the term “disease or disorder involving cartilage degeneration” is any disease or disorder involving cartilage and/or joint degeneration. The term “disease or disorder involving cartilage degeneration” includes conditions, disorders, syndromes, diseases, and injuries that affect spinal discs or joints (e.g., articular joints) in animals, including humans, and includes, but is not limited to, arthritis, chondrophasia, spondyloarthropathy, ankylosing spondylitis, lupus erythematosus, relapsing polychondritis, and Sjogren's syndrome.

As used herein, the term “muscle degeneration disease or disorder” is any disease or disorder involving muscle degeneration. The term includes conditions, disorders, syndromes, diseases, and injuries that affect muscle tissue such as, but not limited to, muscle atrophy, muscle disuse, muscle tears, burns, surgery, peripheral neuropathy, multiple sclerosis, amyotrophic lateral sclerosis (ALS), Duchenne muscular dystrophy, primary lateral sclerosis, myasthenia gravis, cancer, AIDS, congestive heart failure, chronic obstructive pulmonary disease (COPD), liver disease, renal failure, eating disorders, malnutrition, starvation, infections, or treatment with glucocorticoids.

By “therapeutically effective dose or amount” is intended an amount of rejuvenated cells or non-integrative messenger RNAs that brings about a positive therapeutic response in a subject in need of tissue repair or regeneration, such as an amount that restores function and/or results in the generation of new tissue at a treatment site. The rejuvenated cells may be produced by transfection in vitro, ex vivo, or in vivo with one or more non-integrative messenger RNAs encoding one or more cellular reprogramming factors, as described herein. Thus, for example, a “positive therapeutic response” would be an improvement in the age-related disease or condition in association with the therapy, and/or an improvement in one or more symptoms of the age-related disease or condition in association with the therapy, such as restored tissue functionality, reduced pain, improved stamina, increased strength, increased mobility, and/or improved cognitive function. The exact amount (of cells or mRNA) required will vary from subject to subject, depending on the species, age, and general condition of the subject, the severity of the condition being treated, mode of administration, and the like. An appropriate “effective” amount in any individual case may be determined by one of ordinary skill in the art using routine experimentation, based upon the information provided herein.

For example, a therapeutically effective dose or amount of rejuvenated chondrocytes is intended an amount that, when administered as described herein, brings about a positive therapeutic response in a subject having cartilage damage or loss, such as an amount that results in the generation of new cartilage at a treatment site (e.g., a damaged joint). For example, a therapeutically effective dose or amount could be used to treat cartilage damage or loss resulting from a traumatic injury or a degenerative disease, such as arthritis or other disease involving cartilage degeneration. Preferably, a therapeutically effective amount restores function and/or relieves pain and inflammation associated with cartilage damage or loss.

In another example, a therapeutically effective dose or amount of rejuvenated skeletal muscle stem cells is intended an amount that, when administered as described herein, brings about a positive therapeutic response in a subject having muscle damage or loss, such as an amount that results in the generation of new myofibers at a treatment site (e.g., a damaged muscle). For example, a therapeutically effective dose or amount could be used to treat muscle damage or loss resulting from a traumatic injury or a disease or disorder involving muscle degeneration. Preferably, a therapeutically effective amount improves muscle strength and function.

As used herein, the terms “subject,” “individual,” and “patient,” are used interchangeably herein and refer to any vertebrate subject, including, without limitation, humans and other primates, including non-human primates such as chimpanzees and other apes and monkey species; farm animals such as cattle, sheep, pigs, goats and horses; domestic mammals such as dogs and cats; rodents such as mice, rats, rabbits, hamsters, and guinea pigs; birds, including domestic, wild and game birds such as chickens, turkeys and other gallinaceous birds, ducks, geese, and the like. In some cases, the methods of the disclosure find use in experimental animals, in veterinary application, and in the development of animal models for disease. The term does not denote a particular age. Thus, both adult and newborn individuals are intended to be covered.

### II. Methods

Before describing the present disclosure in detail, it is to be understood that this disclosure is not limited to particular formulations or process parameters as such may, of course, vary. It is also to be understood that the terminology used herein is for the purpose of describing particular embodiments of the disclosure only, and is not intended to be limiting.

Although a number of methods and materials similar or equivalent to those described herein can be used in the practice of the present disclosure, the preferred materials and methods are described herein.

The present disclosure relates to methods of rejuvenating aged cells and tissue to restore functionality by transient overexpression of mRNAs affecting, for example, mitochondrial function, proteolytic activity, heterochromatin levels, histone methylation, nuclear lamina polypeptides, cytokine secretion, or senescence. In particular, the inventors have shown that mRNAs encoding OCT4, SOX2, KLF4, c-MYC, LIN28 and NANOG can be used to rejuvenate a variety of cell types, including fibroblasts, endothelial cells, chondrocytes, and skeletal muscle stem cells while retaining cells in a differentiated cell state.

In order to further an understanding of the disclosure, a more detailed discussion is provided below regarding methods of rejuvenating cells by transient reprogramming with mRNAs and cell-based therapies using such rejuvenated cells.

a. Rejuvenating Cells

In an aspect, provided herein are methods of rejuvenating cells, the methods including transfecting cells with one or more non-integrative messenger RNAs encoding one or more cellular reprogramming factors for not more than five (5) continuous days, thereby producing rejuvenated cells.

In embodiments, the rejuvenated cells have a phenotype or activity profile similar to a young cell. The phenotype or activity profile includes one or more of the transcriptomic profile, gene expression of one or more nuclear and/or epigenetic markers, proteolytic activity, mitochondrial health and function, SASP cytokine expression, and methylation landscape.

In embodiments, the rejuvenated cells have a transcriptomic profile that is more similar to the transcriptomic profile of young cells. In embodiments, the transcriptomic profile of the rejuvenated cells includes an increase in gene expression of one or more genes selected from RPL37, RHOA, SRSF3, EPHB4, ARHGAP18, RPL31, FKBP2, MAP1LC3B2, Elf1, Phf8, Pol2s2, Taf1 and Sin3a. In embodiments, the transcriptomic profile of the rejuvenated cells includes an increase in gene expression of RPL37. In embodiments, the transcriptomic profile of the rejuvenated cells includes an increase in gene expression of RHOA. In embodiments, the transcriptomic profile of the rejuvenated cells includes an increase in gene expression of SRSF3. In embodiments, the transcriptomic profile of the rejuvenated cells includes an increase in gene expression of EPHB4. In embodiments, the transcriptomic profile of the rejuvenated cells includes an increase in gene expression of ARHGAP18. In embodiments, the transcriptomic profile of the rejuvenated cells includes an increase in gene expression of RPL31. In embodiments, the transcriptomic profile of the rejuvenated cells includes an increase in gene expression of FKBP2. In embodiments, the transcriptomic profile of the rejuvenated cells includes an increase in gene expression of MAP1LC3B2. In embodiments, the transcriptomic profile of the rejuvenated cells includes an increase in gene expression of Elf1. In embodiments, the transcriptomic profile of the rejuvenated cells includes an increase in gene expression of Phf8. In embodiments, the transcriptomic profile of the rejuvenated cells includes an increase in gene expression of Pol2s2. In embodiments, the transcriptomic profile of the rejuvenated cells includes an increase in gene expression of Taf1. In embodiments, the transcriptomic profile of the rejuvenated cells includes an increase in gene expression of Sin3a. In embodiments, the transcriptomic profile of the rejuvenated cells includes an increase in gene expression of RPL37, RHOA, SRSF3, EPHB4, ARHGAP18, RPL31, FKBP2, MAP1LC3B2, Elf1, Phf8, Pol2s2, Taf1 and Sin3a.

In embodiments, the rejuvenated cells exhibit increased gene expression of one or more nuclear and/or epigenetic markers compared to a reference value. In embodiments, the one or more nuclear and/or epigenetic markers is selected from HP1gamma, H3K9me3, lamina support protein LAP2alpha, and SIRT1 protein. In embodiments, the rejuvenated cells exhibit increased gene expression of HP1gamma. In embodiments, the rejuvenated cells exhibit increased gene expression of H3K9me3. In embodiments, the rejuvenated cells exhibit increased gene expression of lamina support protein LAP2alpha. In embodiments, the rejuvenated cells exhibit increased gene expression of SIRT1 protein. In embodiments, the rejuvenated cells exhibit increased gene expression of HP1gamma, H3K9me3, lamina support protein LAP2alpha, and SIRT1 protein.

In embodiments, the rejuvenated cells have a proteolytic activity that is more similar to the proteolytic activity of young cells. In embodiments, the proteolytic activity is measured as increased cell autophagosome formation, increased chymotrypsin-like proteasome activity, or a combination thereof. In embodiments, the proteolytic activity is measured as increased cell autophagosome formation. In embodiments, the proteolytic activity is measured as increased chymotrypsin-like proteasome activity. In embodiments, the proteolytic activity is measured as increased cell autophagosome formation and increased chymotrypsin-like proteasome activity.

In embodiments, the rejuvenated cells exhibit improved mitochondria health and function compared to a reference value. In embodiments, improved mitochondria health and function is measured as increased mitochondria membrane potential, decreased reactive oxygen species (ROS), or a combination thereof. In embodiments, improved mitochondria health and function is measured as increased mitochondria membrane potential. In embodiments, improved mitochondria health and function is measured as decreased reactive oxygen species (ROS). In embodiments, improved mitochondria health and function is measured as increased mitochondria membrane potential and decreased reactive oxygen species (ROS).

In embodiments, the rejuvenated cells exhibit decreased expression of one or more SASP cytokines compared to a reference value. In embodiments, the one or more SASP cytokines include IL18, IL1A, GROA, IL22, and IL9. In embodiments, the rejuvenated cells exhibit decreased expression of IL18. In embodiments, the rejuvenated cells exhibit decreased expression of IL1A. In embodiments, the rejuvenated cells exhibit decreased expression of GROA. In embodiments, the rejuvenated cells exhibit decreased expression of IL22. In embodiments, the rejuvenated cells exhibit decreased expression of IL9. In embodiments, the rejuvenated cells exhibit decreased expression of IL18, IL1A, GROA, IL22, and IL9.

In embodiments, the rejuvenated cells exhibit reversal of the methylation landscape. In embodiments, the reversal of the methylation landscape is measured by Horvath clock estimation.

In embodiments, a reference value is obtained from an aged cell.

In embodiments, cells are rejuvenated by transient reprogramming with mRNAs encoding one or more cellular reprogramming factors. Transient reprogramming is accomplished by transfecting cells once daily with non-integrative mRNAs for at least two days and not more than 5 days. By “non-integrative” is meant that a mRNA molecule is not integrated intrachromosomally or extrachromosomally into the host genome, nor integrated into a vector, such that reprogramming is transient and does not destroy the identity of the rejuvenated cell (i.e., cell retains capability of differentiating into its adult cell-type). In embodiments, transient reprogramming of cells eliminates various hallmarks of aging while avoiding complete dedifferentiation of the cells into stem cells.

In embodiments, transfecting cells with messenger RNAs may be accomplished by a transfection method selected from lipofectamine and LT-1 mediated transfection, dextran-mediated transfection, calcium phosphate precipitation, polybrene mediated transfection, electroporation, encapsulation of the mRNAs in liposomes, and direct microinjection. In embodiments, transfecting cells with messenger RNAs may be accomplished by lipofectamine and LT-1 mediated transfection. In embodiments, transfecting cells with messenger RNAs may be accomplished by dextran-mediated transfection. In embodiments, transfecting cells with messenger RNAs may be accomplished by calcium phosphate precipitation. In embodiments, transfecting cells with messenger RNAs may be accomplished by polybrene mediated transfection. In embodiments, transfecting cells with messenger RNAs may be accomplished by electroporation. In embodiments, transfecting cells with messenger RNAs may be accomplished by encapsulation of the mRNAs in liposomes. In embodiments, transfecting cells with messenger RNAs may be accomplished by direct microinjection.

Cellular age-reversal, or rejuvenating, is achieved by transient overexpression of one or more mRNAs encoding cellular reprogramming factors. Such cellular reprogramming factors may include transcription factors, epigenetic remodelers, or small molecules affecting mitochondrial function, proteolytic activity, heterochromatin levels, histone methylation, nuclear lamina polypeptides, cytokine secretion, or senescence. In In embodiments, the cellular reprogramming factors include one or more of OCT4, SOX2, KLF4, c-MYC, LIN28 and NANOG. In another embodiment, the cellular reprogramming factors include OCT4, SOX2, KLF4, c-MYC, LIN28 and NANOG. In certain embodiments, the cellular reprogramming factors consist of OCT4, SOX2, KLF4, c-MYC, LIN28 and NANOG.

In embodiments, the methods provided herein may be applied to any type of cell in need of rejuvenation. The cell may be isolated from other cells, mixed with other cells in a culture, or within a tissue (partial or intact), or a live organism. The methods described herein can be performed, for example, on a sample comprising a single cell, a population of cells, or a tissue or organ comprising cells. The cells chosen for rejuvenation will depend on the desired therapeutic effect for treating an age-related disease or condition.

In embodiments, the cells are mammalian cells. In embodiments, the cells are human cells. In embodiments, the cells are from an elderly subject.

In embodiments, the methods provided herein may be performed on cells, tissue, or organs of the nervous system, muscular system, respiratory system, cardiovascular system, skeletal system, reproductive system, integumentary system, lymphatic system, excretory system, endocrine system (e.g. endocrine and exocrine), or digestive system. Any type of cell can potentially be rejuvenated, as described herein, including, but not limited to, epithelial cells (e.g., squamous, cuboidal, columnar, and pseudostratified epithelial cells), endothelial cells (e.g., vein, artery, and lymphatic vessel endothelial cells), and cells of connective tissue, muscles, and the nervous system. Such cells may include, but are not limited to, epidermal cells, fibroblasts, chondrocytes, skeletal muscle cells, satellite cells, heart muscle cells, smooth muscle cells, keratinocytes, basal cells, ameloblasts, exocrine secretory cells, myoepithelial cells, osteoblasts, osteoclasts, neurons (e.g., sensory neurons, motor neurons, and interneurons), glial cells (e.g., oligodendrocytes, astrocytes, ependymal cells, microglia, Schwann cells, and satellite cells), pillar cells, adipocytes, pericytes, stellate cells, pneumocytes, blood and immune system cells (e.g., erythrocytes, monocytes, dendritic cells, macrophages, neutrophils, eosinophils, mast cells, T cells, B cells, natural killer cells), hormone-secreting cells, germ cells, interstitial cells, lens cells, photoreceptor cells, taste receptor cells, and olfactory cells; as well as cells and/or tissue from the kidney, liver, pancreas, stomach, spleen, gall bladder, intestines, bladder, lungs, prostate, breasts, urogenital tract, pituitary cells, oral cavity, esophagus, skin, hair, nail, thyroid, parathyroid, adrenal gland, eyes, nose, or brain.

In some embodiments, the cells are selected from fibroblasts, endothelial cells, chondrocytes, skeletal muscle stem cells, keratinocytes, mesenchymal stem cells and corneal epithelial cells.

In embodiments, the cells are fibroblasts. In embodiments, the cells are endothelial cells. In embodiments, the cells are chondrocytes. In embodiments, the cells are skeletal muscle stem cells. In embodiments, the cells are keratinocytes. In embodiments, the cells are mesenchymal stem cells. In embodiments, the cells are corneal epithelial cells.

In embodiments, the rejuvenated fibroblasts exhibit a transcriptomic profile similar to a transcriptomic profile of young fibroblasts. In embodiments, the rejuvenated fibroblasts exhibit an increased gene expression of one or more nuclear and/or epigenetic markers compared to a reference value as described above. In embodiments, the rejuvenated fibroblasts have a proteolytic activity that is more similar to the proteolytic activity of young cells as described above. In embodiments, the rejuvenated fibroblasts exhibit improved mitochondria health and function compared to a reference value as described above. In embodiments, the rejuvenated fibroblasts exhibit a reversal of the methylation landscape.

In embodiments, the rejuvenated endothelial cells exhibit a transcriptomic profile similar to a transcriptomic profile of young endothelial cells. In embodiments, the rejuvenated endothelial cells exhibit increased gene expression of one or more nuclear and/or epigenetic markers compared to a reference value as described above. In embodiments, the rejuvenated endothelial cells have a proteolytic activity that is more similar to the proteolytic activity of young cells as described above. In embodiments, the rejuvenated endothelial cells exhibit improved mitochondria health and function compared to a reference value as described above. In embodiments, the rejuvenated endothelial cells exhibit a reversal of the methylation landscape.

In embodiments, the rejuvenated chondrocytes exhibit reduced expression of inflammatory factors and/or and increased ATP and collagen metabolism. In embodiments, the inflammatory factors include RANKL, iNOS2, IL6, IFNα, MCP3 and MIP1A. In embodiments, the rejuvenated chondrocytes exhibit reduced expression of RANKL. In embodiments, the rejuvenated chondrocytes exhibit reduced expression of iNOS2. In embodiments, the rejuvenated chondrocytes exhibit reduced expression of IL6. In embodiments, the rejuvenated chondrocytes exhibit reduced expression of IFNα. In embodiments, the rejuvenated chondrocytes exhibit reduced expression of MCP3. In embodiments, the rejuvenated chondrocytes exhibit reduced expression of MIP1A. In embodiments, the rejuvenated chondrocytes exhibit reduced expression of RANKL, iNOS2, IL6, IFNα, MCP3 and MIP1A. In embodiments, the rejuvenated chondrocytes exhibit increased ATP and collagen metabolism. In embodiments, ATP and collagen metabolism is measured by one or more of increased ATP levels, decreased ROS and increased SOD2 expression, increased COL2A1 expression and overall proliferation by the chondrocytes. In embodiments, ATP and collagen metabolism is measured by increased ATP levels. In embodiments, ATP and collagen metabolism is measured by decreased ROS and increased SOD2 expression. In embodiments, ATP and collagen metabolism is measured by increased COL2A1 expression and overall proliferation by the chondrocytes.

In embodiments, the rejuvenated skeletal muscle stem cells exhibit higher proliferative capacity, enhanced ability to differentiate into myoblasts and muscle fibers, restored lower kinetics of activation from quiescence, ability to rejuvenate the muscular microniche, restore youthful force in the muscle, or a combination thereof.

In embodiments, the rejuvenated keratinocytes exhibit higher proliferative capacity, reduced inflammatory phenotype, lower RNAKL and INOS2 expression, reduced expression of cytokines MIP1A, IL6, IFNα, MCP3, increased ATP, increased levels of SOD2 and COL2A1 expression.

In embodiments, the rejuvenated mesenchymal stem cells exhibit reduction in senescence parameters, increased cell proliferation, and/or a decrease in ROS levels. In embodiments, the rejuvenated mesenchymal stem cells exhibit reduction in senescence parameters. In embodiments, the senescence parameters include p16 expression, p21 expression and positive SAβGal staining. In embodiments, the rejuvenated mesenchymal stem cells exhibit increased cell proliferation. In embodiments, the rejuvenated mesenchymal stem cells exhibit a decrease in ROS levels. In embodiments, the rejuvenated mesenchymal stem cells exhibit reduction in senescence parameters, increased cell proliferation, and a decrease in ROS levels.

In embodiments, the rejuvenated corneal epithelial cells exhibit a reduction in senescence parameters. In embodiments, the senescence parameters include one or more of expression of p21, expression of p16, mitochondria biogenesis PGC1α, and expression of inflammatory factor IL8. In embodiments, the senescence parameters include p21. In embodiments, the senescence parameters include expression of p16. In embodiments, the senescence parameters include mitochondria biogenesis PGC1α. In embodiments, the senescence parameters include expression of inflammatory factor IL8. In embodiments, the senescence parameters include one expression of p21, expression of p16, mitochondria biogenesis PGC1α, and expression of inflammatory factor IL8.

The methods of the disclosure can be used to rejuvenate cells in culture (e.g., ex vivo or in vitro) to improve function and potency for use in cell therapy. The cells used in treatment of a patient may be autologous or allogeneic. Preferably, the cells are derived from the patient or a matched donor. For example, in ex vivo therapy, cells are obtained directly from the patient to be treated, transfected with mRNAs encoding cellular reprogramming factors, as described herein, and reimplanted in the patient. Such cells can be obtained, for example, from a biopsy or surgical procedure performed on the patient. Alternatively, cells in need of rejuvenation can be transfected directly in vivo with mRNAs encoding cellular reprogramming factors.

Transfection may be performed using any suitable method known in the art that provides for transient uptake of mRNAs encoding cellular reprogramming factors into cells in need of rejuvenation (i.e., for transient reprogramming). In embodiments, methods for ex vivo, in vitro, or in vivo delivery of mRNA into cells of a subject can include a method selected from lipofectamine and LT-1 mediated transfection, dextran-mediated transfection, calcium phosphate precipitation, polybrene mediated transfection, electroporation, encapsulation of the mRNAs in liposomes, direct microinjection of the mRNAs into cells, or a combination thereof.

b. Compositions

In an aspect, provided herein are pharmaceutical compositions including rejuvenated cells obtained by transfecting cells with one or more non-integrative messenger RNAs encoding one or more cellular reprogramming factors for not more than five (5) continuous days.

In embodiments, the rejuvenated cells are autologous. In embodiments, the rejuvenated cells are allogeneic.

In embodiments, the one or more cellular reprogramming factors are selected from OCT4, SOX2, KLF4, c-MYC, LIN28 and NANOG. In embodiments, the cellular reprogramming factors are OCT4, SOX2, KLF4, c-MYC, LIN28 and NANOG.

In embodiments, the rejuvenated cells display one or more of the following: increased expression of HP1 gamma, H3K9me3, LAP2alpha, SIRT1, increased mitochondrial membrane potential and decreased reactive oxygen species, and decreased expression of SASP cytokines. In embodiments, SASP cytokines include one or more of IL18, IL1A, GROA, IL22, and IL9.

In certain embodiments, compositions comprising rejuvenated cells for use in cell therapy may further comprise one or more additional factors, such as nutrients, cytokines, growth factors, extracellular matrix (ECM) components, antibiotics, anti-oxidants, or immunosuppressive agents to improve cell function or viability. The composition may also further comprise a pharmaceutically acceptable carrier.

Examples of growth factors include, but are not limited to, fibroblast growth factor (FGF), insulin-like growth factor (IGF), transforming growth factor beta (TGF-β), epiregulin, epidermal growth factor (“EGF”), endothelial cell growth factor (“ECGF”), nerve growth factor (“NGF”), leukemia inhibitory factor (“LIF”), bone morphogenetic protein-4 (“BMP-4”), hepatocyte growth factor (“HGF”), vascular endothelial growth factor-A (“VEGF-A”), and cholecystokinin octapeptide.

Examples of ECM components include, but are not limited to, proteoglycans (e.g., chondroitin sulfate, heparan sulfate, and keratan sulfate), non-proteoglycan polysaccharides (e.g., hyaluronic acid), fibers (e.g., collagen and elastin), and other ECM components (e.g., fibronectin and laminin).

Examples of immunosuppressive agents include, but are not limited to, steroidal (e.g., prednisone) or non-steroidal (e.g., sirolimus (Rapamune, Wyeth-Ayerst Canada), tacrolimus (Prograf, Fujisawa Canada), and anti-IL2R daclizumab (Zenapax, Roche Canada). Other immunosuppressant agents include 15-deoxyspergualin, cyclosporin, methotrexate, rapamycin, Rapamune (sirolimus/rapamycin), FK506, or Lisofylline (LSF).

One or more pharmaceutically acceptable excipients may also be included. Examples include, but are not limited to, carbohydrates, inorganic salts, antimicrobial agents, antioxidants, surfactants, buffers, acids, bases, and combinations thereof.

For example, an antimicrobial agent for preventing or deterring microbial growth may be included. Non-limiting examples of antimicrobial agents suitable for the present disclosure include benzalkonium chloride, benzethonium chloride, benzyl alcohol, cetylpyridinium chloride, chlorobutanol, phenol, phenylethyl alcohol, phenylmercuric nitrate, thimersol, and combinations thereof. Antimicrobial agents also include antibiotics that can also be used to prevent bacterial infection. Examples antibiotics include amoxicillin, penicillin, sulfa drugs, cephalosporins, erythromycin, streptomycin, gentamicin, tetracycline, chlarithromycin, ciproflozacin, azithromycin, and the like. Also included are antifungal agents such as myconazole and terconazole.

Various antioxidants can also be included, such as molecules having thiol groups such as reduced glutathione (GSH) or its precursors, glutathione or glutathione analogs, glutathione monoester, and N-acetylcysteine. Other suitable anti-oxidants include superoxide dismutase, catalase, vitamin E, Trolox, lipoic acid, lazaroids, butylated hydroxyanisole (BHA), vitamin K, and the like.

Excipients suitable for injectable compositions include water, alcohols, polyols, glycerin, vegetable oils, phospholipids, and surfactants. A carbohydrate such as a sugar, a derivatized sugar such as an alditol, aldonic acid, an esterified sugar, and/or a sugar polymer may be present as an excipient. Specific carbohydrate excipients include, for example: monosaccharides, such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol, sorbitol (glucitol), pyranosyl sorbitol, myoinositol, and the like. The excipient can also include an inorganic salt or buffer such as citric acid, sodium chloride, potassium chloride, sodium sulfate, potassium nitrate, sodium phosphate monobasic, sodium phosphate dibasic, and combinations thereof.

Acids or bases can also be present as an excipient. Non-limiting examples of acids that can be used include those acids selected from the group consisting of hydrochloric acid, acetic acid, phosphoric acid, citric acid, malic acid, lactic acid, formic acid, trichloroacetic acid, nitric acid, perchloric acid, phosphoric acid, sulfuric acid, fumaric acid, and combinations thereof. Examples of suitable bases include, without limitation, bases selected from the group consisting of sodium hydroxide, sodium acetate, ammonium hydroxide, potassium hydroxide, ammonium acetate, potassium acetate, sodium phosphate, potassium phosphate, sodium citrate, sodium formate, sodium sulfate, potassium sulfate, potassium fumerate, and combinations thereof.

Typically, the optimal amount of any individual excipient is determined through routine experimentation, i.e., by preparing compositions containing varying amounts of the excipient (ranging from low to high), examining the stability and other parameters, and then determining the range at which optimal performance is attained with no significant adverse effects. Generally, however, the excipient(s) will be present in the composition in an amount of about 1% to about 99% by weight, preferably from about 5% to about 98% by weight, more preferably from about 15 to about 95% by weight of the excipient, with concentrations less than 30% by weight most preferred. These foregoing pharmaceutical excipients along with other excipients are described in “Remington: The Science & Practice of Pharmacy”, 19th ed., Williams & Williams, (1995), the “Physician's Desk Reference”, 52nd ed., Medical Economics, Montvale, NJ (1998), and Kibbe, A. H., Handbook of Pharmaceutical Excipients, 3rd Edition, American Pharmaceutical Association, Washington, D.C., 2000.

c. Administration

The methods of the disclosure can be used for treating a subject for an age-related disease or condition. For example, cell therapies involving transient reprogramming of cells by transfection with non-integrated mRNAs encoding reprograming factors (e.g., in vitro, ex vivo, or in vivo) can be used for treating a subject for a variety of age-related diseases and conditions such as, but not limited to, neurodegenerative diseases (e.g., Alzheimer's disease, Parkinson's disease, Huntington's disease, amyotrophic lateral sclerosis, dementia, and stroke), cardiovascular and peripheral vascular diseases (e.g., atherosclerosis, peripheral arterial disease (PAD), hematomas, calcification, thrombosis, embolisms, and aneurysms), eye diseases (e.g., age-related macular degeneration, glaucoma, cataracts, dry eye, diabetic retinopathy, vision loss), dermatologic diseases (dermal atrophy and thinning, elastolysis and skin wrinkling, sebaceous gland hyperplasia or hypoplasia, senile lentigo and other pigmentation abnormalities, graying hair, hair loss or thinning, and chronic skin ulcers), autoimmune diseases (e.g., polymyalgia rheumatica (PMR), giant cell arteritis (GCA), rheumatoid arthritis (RA), crystal arthropathies, and spondyloarthropathy (SPA)), endocrine and metabolic dysfunction (e.g., adult hypopituitarism, hypothyroidism, apathetic thyrotoxicosis, osteoporosis, diabetes mellitus, adrenal insufficiency, various forms of hypogonadism, and endocrine malignancies), musculoskeletal disorders (e.g., arthritis, osteoporosis, myeloma, gout, Paget's disease, bone fractures, bone marrow failure syndrome, ankylosis, diffuse idiopathic skeletal hyperostosis, hematogenous osteomyelitis, muscle atrophy, peripheral neuropathy, multiple sclerosis, amyotrophic lateral sclerosis (ALS), Duchenne muscular dystrophy, primary lateral sclerosis, and myasthenia gravis), diseases of the digestive system (e.g., liver cirrhosis, liver fibrosis, Barrett's esophagus), respiratory diseases (e.g., pulmonary fibrosis, chronic obstructive pulmonary disease, asthma, chronic bronchitis, pulmonary embolism (PE), lung cancer, and infections), and any other diseases and disorders associated with aging.

In an aspect, provided herein are method for treating a subject for an age-related disease or condition, a cartilage degeneration disorder, a neurodegenerative disorder, and/or musculoskeletal dysfunction. The methods include administering a therapeutically effective amount of cells that include one or more non-integrative messenger RNAs encoding one or more cellular reprogramming factors.

At least one therapeutically effective cycle of treatment by transfection with one or more non-integrative messenger RNAs encoding one or more cellular reprogramming factors may be administered to a subject for treatment of an age-related disease or condition.

In embodiments, the age-related disease or condition is selected from an eye, skin, or musculoskeletal dysfunction.

In embodiments, the subject has a cartilage degeneration disorder. In embodiments, the disorder is selected from arthritis, chondrophasia, spondyloarthropathy, ankylosing spondylitis, lupus erythematosus, relapsing polychondritis, and Sjogren's syndrome. In embodiments, the disorder is arthritis. In embodiments, the disorder is chondrophasia. In embodiments, the disorder is spondyloarthropathy. In embodiments, the disorder is ankylosing spondylitis. In embodiments, the disorder is lupus erythematosus. In embodiments, the disorder is relapsing polychondritis, In embodiments, the disorder is Sjogren's syndrome.

In embodiments, treating reduces expression of one or more inflammatory factors and/or and increases ATP and collagen metabolism. In embodiments, the inflammatory factors are selected from RANKL, iNOS2, IL6, IFNα, MCP3 and MIP1A. In embodiments, ATP and collagen metabolism is measured by one or more of increased ATP levels, decreased ROS and increased SOD2, increased COL2A1 and overall proliferation by the chondrocytes

In embodiments, treatment of a subject with cells rejuvenated by transfection ex vivo or in vitro in cell culture, compositions for transplanting rejuvenated cells are typically, though not necessarily, administered by injection or surgical implantation into a region requiring tissue regeneration or repair.

In embodiments, the therapeutically effective amount of rejuvenated cells is selected from fibroblasts, endothelial cells, chondrocytes, skeletal muscle stem cells, keratinocytes, mesenchymal stem cells and corneal epithelial cells. In embodiments, the therapeutically effective amount of rejuvenated cells are fibroblasts. In embodiments, the therapeutically effective amount of rejuvenated cells are endothelial cells. In embodiments, the therapeutically effective amount of rejuvenated cells are chondrocytes. In embodiments, the therapeutically effective amount of rejuvenated cells are skeletal muscle stem cells. In embodiments, the therapeutically effective amount of rejuvenated cells are keratinocytes. In embodiments, the therapeutically effective amount of rejuvenated cells are mesenchymal stem cells. In embodiments, the therapeutically effective amount of rejuvenated cells are corneal epithelial cells.

In embodiments, the rejuvenated corneal epithelial exhibit a reduction in senescence parameters. In embodiments, the senescence parameters include one or more of expression of p21 and p16, mitochondria biogenesis PGC1α, and expression of inflammatory factor IL8.

In one embodiment, chondrocytes in an area of cartilage damage or loss are transfected in vivo with an effective amount of one or more non-integrative messenger RNAs encoding one or more cellular reprogramming factors sufficient to result in rejuvenation of chondrocytes and generation of new cartilage at a treatment site. Alternatively, rejuvenated chondrocytes, produced by transfection ex vivo or in vitro, can be administered locally into an area of cartilage damage or loss, such as a damaged joint or other suitable treatment site of the subject. By therapeutically effective dose or amount of rejuvenated chondrocytes is intended an amount that brings about a positive therapeutic response in a subject having cartilage damage or loss, such as an amount that results in the generation of new cartilage at a treatment site (e.g., a damaged joint). For example, a therapeutically effective dose or amount can be used to treat cartilage damage or loss resulting from a traumatic injury or a degenerative disease, such as arthritis or other disease involving cartilage degeneration. Preferably, a therapeutically effective amount restores function and/or relieves pain and inflammation associated with cartilage damage or loss.

In another embodiment, skeletal muscle stem cells are transfected in vivo with an effective amount of one or more non-integrative messenger RNAs encoding one or more cellular reprogramming factors sufficient to result in rejuvenation (i.e., restore potency) of the skeletal muscle stem cells and generation of new myofibers at a treatment site (e.g., damaged muscle). Alternatively, rejuvenated skeletal muscle stem cells, produced by transfection ex vivo or in vitro, can be administered locally into a damaged muscle in need of repair or regeneration. For example, a therapeutically effective dose or amount could be used to treat muscle damage or loss resulting from a traumatic injury, muscle atrophy, or a disease or disorder involving muscle degeneration. By therapeutically effective dose or amount of rejuvenated skeletal muscle stem cells is intended an amount that brings about a positive therapeutic response in a subject having muscle damage or loss, such as an amount that results in the generation of new myofibers at a treatment site (e.g., a damaged muscle). Preferably, a therapeutically effective amount improves muscle strength and function, relieves pain, improves stamina, and/or increases mobility.

In an aspect, provided herein are methods for treating a subject for an age-related disease or condition, a cartilage degeneration disorder, and/or subject has a musculoskeletal dysfunction as described herein above. The methods include administering a therapeutically effective amount of one or more non-integrative messenger RNAs encoding one or more cellular reprogramming factors, as described herein above.

In embodiments, cells in a subject can be rejuvenated by transfection in vivo with an effective amount of one or more non-integrative messenger RNAs encoding one or more cellular reprogramming factors, as described herein.

In an aspect, provided herein are methods of rejuvenating engineered tissue ex vivo. The methods include transfecting the tissue with one or more non-integrative messenger RNAs encoding one or more cellular reprogramming factors for not more than five (5) continuous days, thereby producing rejuvenated engineered tissue.

In embodiments, the engineered tissue exhibits a reduction in senescence parameters, pro-inflammatory factors, improvements in histological score, or a combination thereof. In embodiments, the engineered tissue exhibits a reduction in one or more senescence parameters. In embodiments, the senescence parameters are selected from p16 expression, positive SaβGal staining, and pro-inflammatory factors IL8 and MMP1 expression. In embodiments, the engineered tissue exhibits a reduction in p16 expression. In embodiments, the engineered tissue exhibits a reduction in positive SaβGal staining. In embodiments, the engineered tissue exhibits a reduction in pro-inflammatory factors IL8 and MMP1 expression. In embodiments, the engineered tissue exhibits, improvements in histological score. In embodiments, the histological score includes morphology, organization, and/or quality.

In embodiments, the engineered tissue is engineered skin tissue and organoids.

d. Kits

The disclosure also provides kits comprising one or more containers holding compositions comprising one or more non-integrative messenger RNAs encoding one or more cellular reprogramming factors for transient reprogramming of cells. Kits may further comprise transfection agents, media for culturing cells, and optionally one or more other factors, such as growth factors, ECM components, antibiotics, and the like. The mRNAs encoding cellular reprogramming factors and/or other compositions can be in liquid form or lyophilized. Such kits may also include components that preserve or maintain the mRNAs that protect against their degradation. Such components may be RNAse-free or protect against RNAses. Suitable containers for the compositions include, for example, bottles, vials, syringes, and test tubes. Containers can be formed from a variety of materials, including glass or plastic. A container may have a sterile access port (for example, the container may be an intravenous solution bag or a vial having a stopper pierceable by a hypodermic injection needle).

The kit can further comprise a second container comprising a pharmaceutically-acceptable buffer, such as phosphate-buffered saline, Ringer's solution, or dextrose solution. It can also contain other materials useful to the end-user, including other pharmaceutically acceptable formulating solutions such as buffers, diluents, filters, needles, and syringes or other delivery devices. The delivery device may be pre-filled with the compositions.

The kit can also comprise a package insert containing written instructions for methods of treating age-related disease or conditions. The package insert can be an unapproved draft package insert or can be a package insert approved by the Food and Drug Administration (FDA) or other regulatory body.

In certain embodiments, the kit comprises mRNAs encoding one or more cellular reprogramming factors selected from the group consisting of OCT4, SOX2, KLF4, c-MYC, LIN28 and NANOG. In one embodiment, the kit comprises mRNAs encoding the OCT4, SOX2, KLF4, c-MYC, LIN28 and NANOG cellular reprogramming factors.

### III. Experimental

Below are examples of specific embodiments for carrying out the present disclosure. The examples are offered for illustrative purposes only, and are not intended to limit the scope of the present disclosure in any way.

Efforts have been made to ensure accuracy with respect to numbers used (e.g., amounts, temperatures, etc.), but some experimental error and deviation should, of course, be allowed for.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### EXAMPLES

### Example 1: Transient and Non-Integrative Cellular Reprogramming Promotes Multifaceted Reversal of Aging

The experiments described herein delineate the degree of age reversal effect that can be achieved by a transient reprogramming protocol, which stops before cell identity is irreversibly lost. Recent evidence has also shown that partial transgenic reprogramming can ameliorate age-associated hallmarks and extend lifespan in progeroid mice. However, it is unknown how this form of ‘epigenetic rejuvenation’ would broadly apply to natural aging and importantly how it could translate safely to human cells. Data herein shows that transient reprogramming based on mRNA technologies reverses hallmarks of physiological aging, reduces age related disease phenotypes and restores regenerative response diminished with age in somatic and stem cells obtained from human clinical samples. The non-integrative method of transient cell reprogramming described herein paves the way to a novel, more translatable, strategy for ex vivo cell rejuvenation treatment aimed towards regenerative medicine and for in vivo tissue rejuvenation therapies to delay or reverse the physiological decay of natural aging and the pathogenesis of age-related diseases.

To test whether any substantial and measurable reprogramming of cellular age can be achieved before a point of no return, and if this can result in any amelioration of cellular function and physiology, the effects of transient reprogramming on the aged physiology of two distinct cell types, fibroblasts and endothelial cells, from otherwise healthy human subjects, was evaluated and compared to the same cell types taken from young donors. Fibroblasts were derived from minced arm and abdomen skin biopsies (25-35 years for the young control, n=3, and 60-70 years for the aged group, n=3) while endothelial cells were extracted from collagenase digest of iliac vein and artery (15-25 years for the young control, n=3, and 45-50 years for the aged group, n=3).

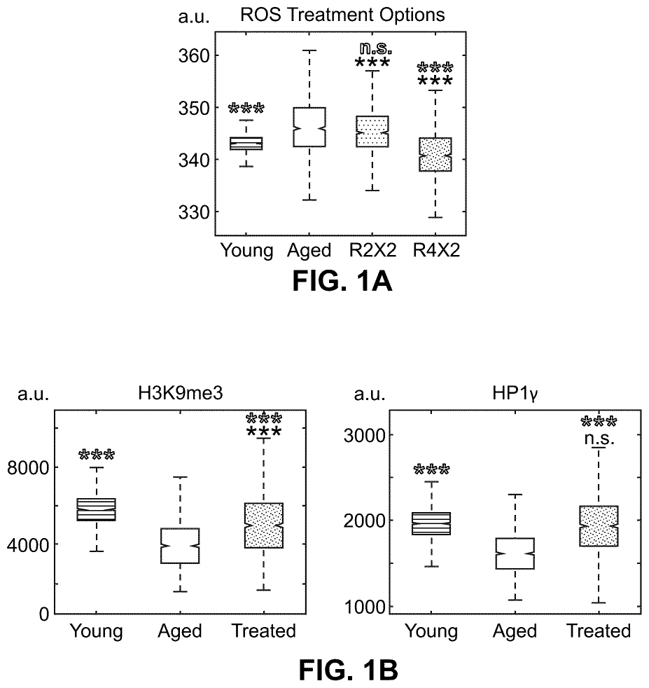

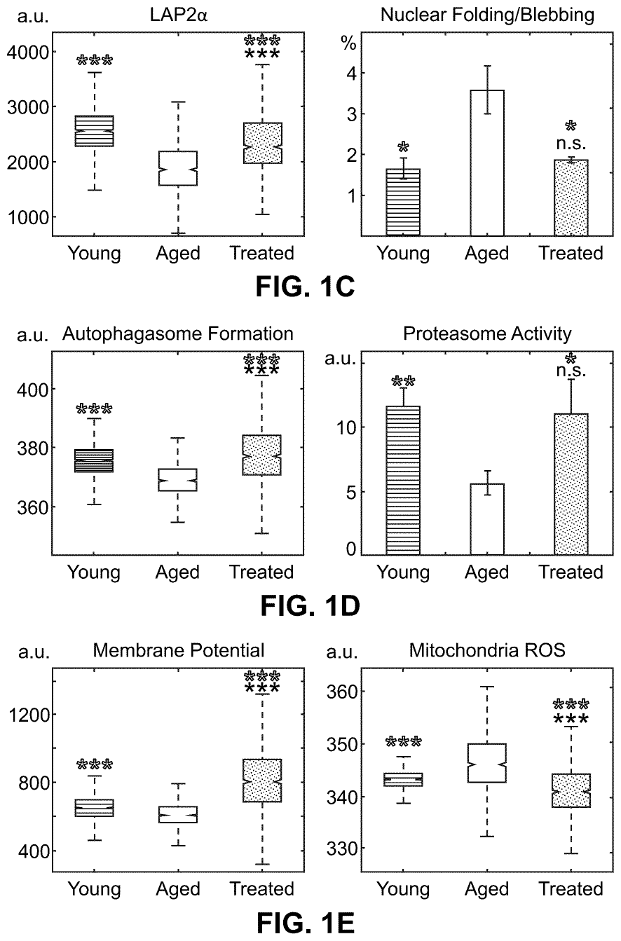

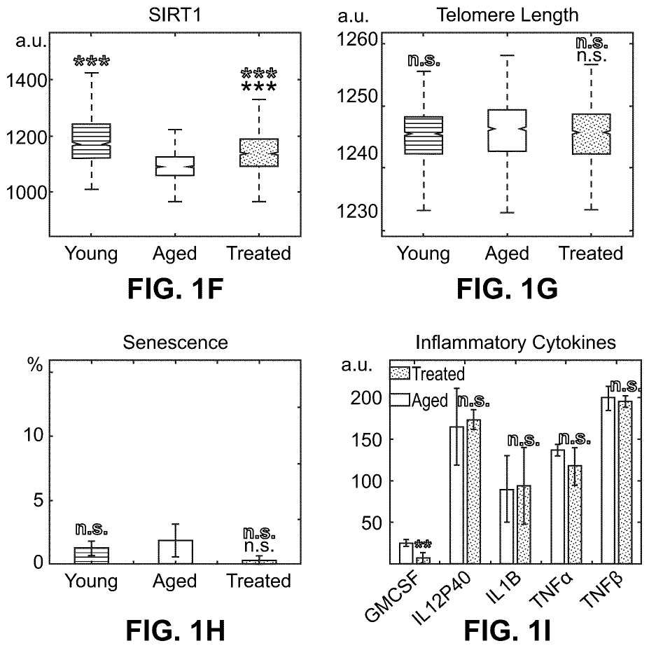

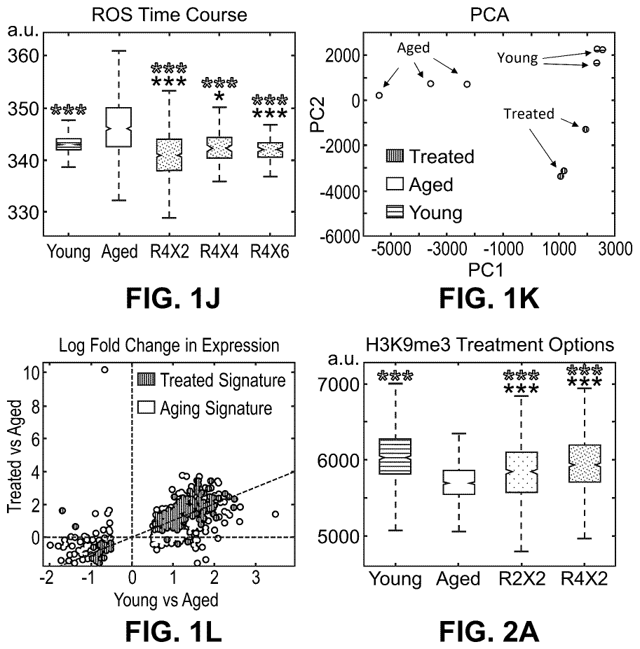

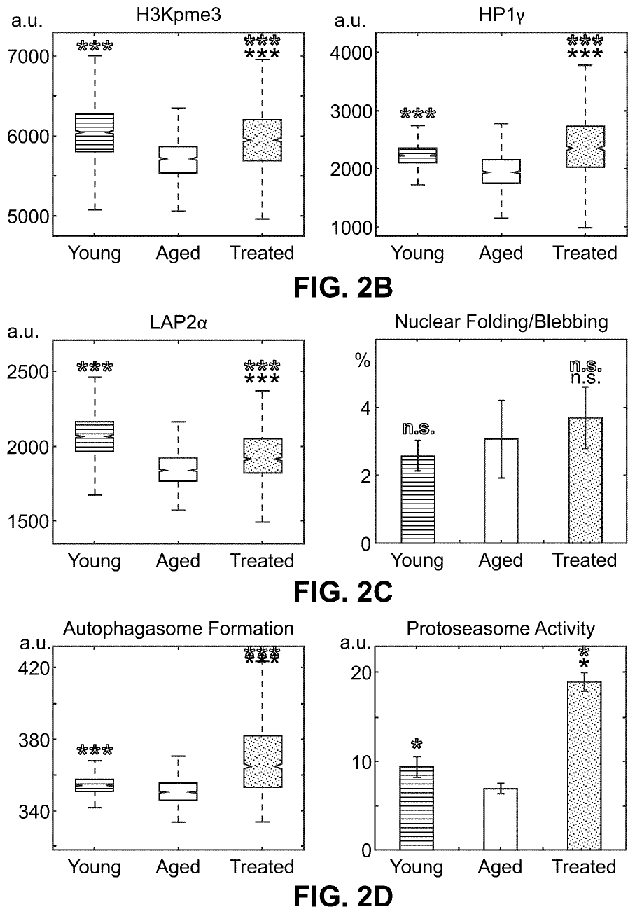

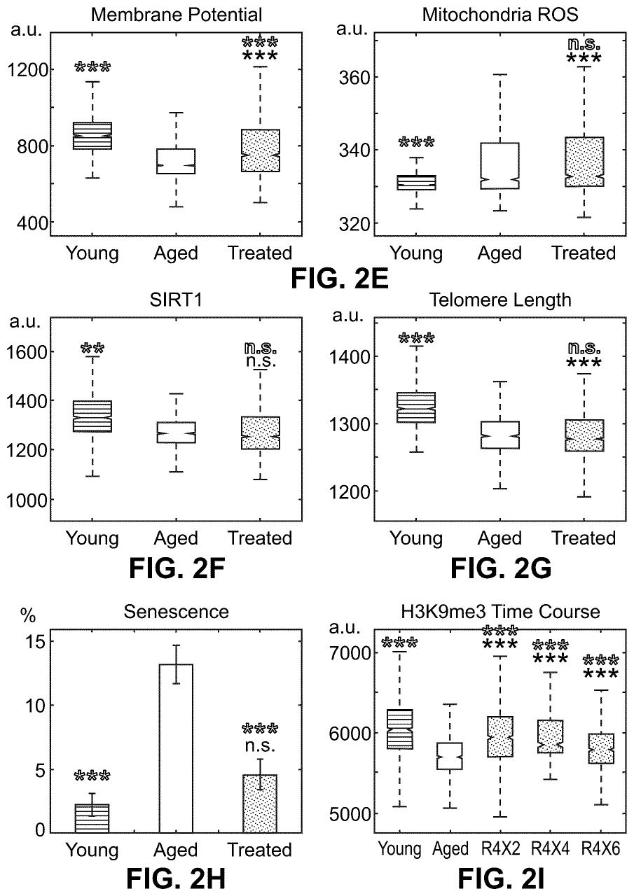

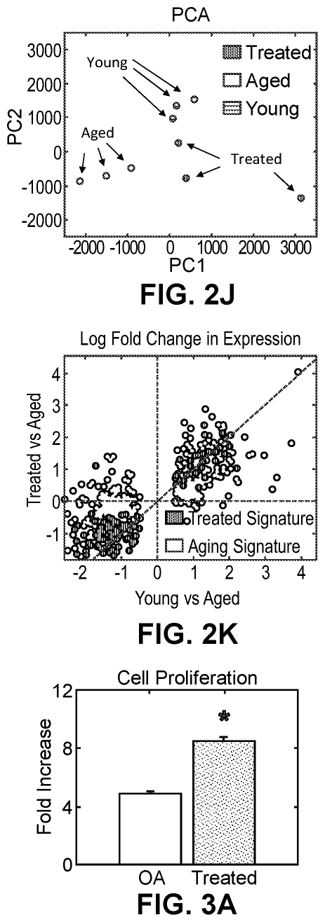

A non-integrative reprogramming protocol was utilized. The protocol was optimized, based on a cocktail of mRNAs expressing OCT4, SOX2, KLF4, c-MYC, LIN28 and NANOG (OSKMLN) was utilized. Multiple reprogramming durations were explored, and while both cell types displayed a rapid change in many aging parameters as early as R2X2 (two reprogramming transfections with two days relaxation back to a basal state), the most pronounced effect was at R4X2 (FIGS. 1A and 2A). The protocol consistently produces induced pluripotent stem cell (iPSC) colonies, regardless of age of the donors, after 12-15 daily transfections; we reasoned that the point of no return PNR in our platform occurs at about day 5 of reprogramming, based on the observation that the first detectable expression of endogenous pluripotency-associated lncRNAs occurs at day 5. Therefore, a transient reprogramming protocol was adopted where OSKMLN were daily transfected for four consecutive days, and performed gene expression analysis two days after the interruption.

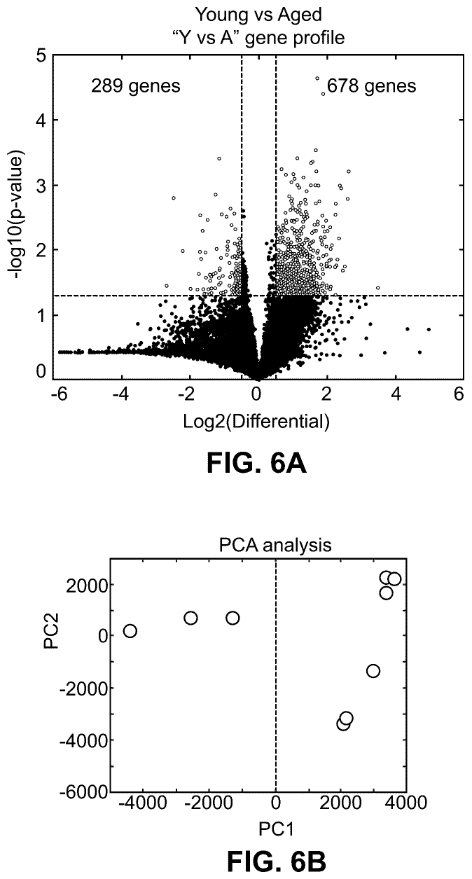

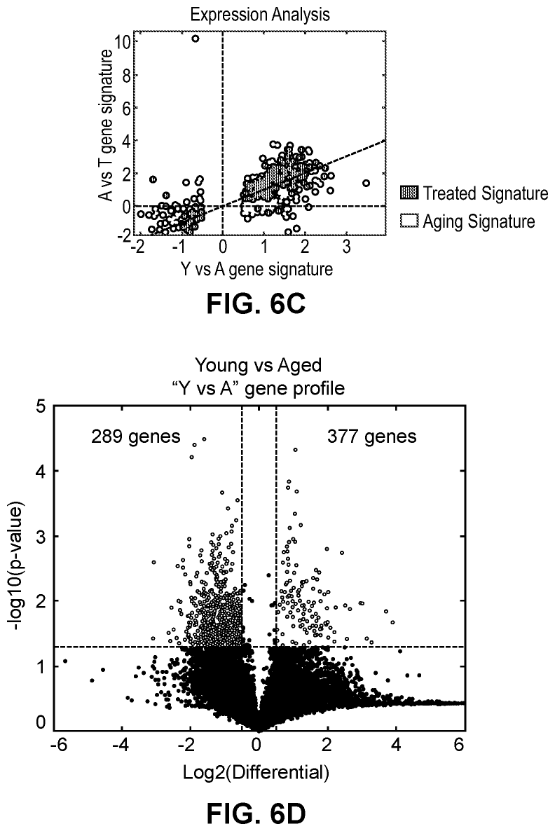

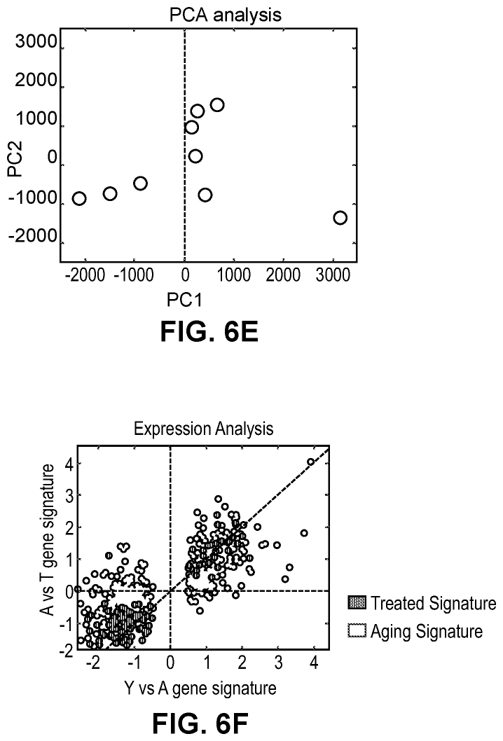

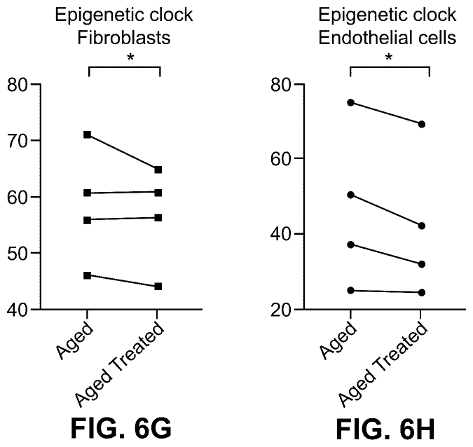

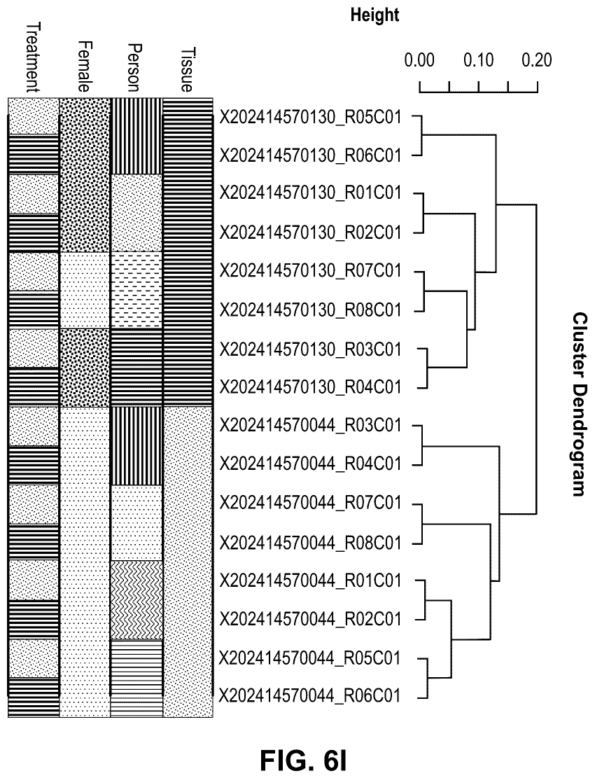

Paired-end bulk RNA sequencing was performed on both cell types for the same three cohorts: young (Y), untreated aged (UA), and treated aged (TA). First, the quantile normalized transcriptomes of young and untreated aged cells for each cell type (“Y vs UA”) was compared. The data shows that 961 genes (5.85%) in fibroblasts (678 upregulated, 289 downregulated) and 748 genes (4.80%) in endothelial cells (389 upregulated, 377 down regulated) differed between young and aged cells, with the significance criteria of p<0.05 and a log fold change cutoff +/−0.5 (FIG. 6). These sets of genes were enriched for many of the known aging pathways, identified in the hallmark gene set collection in the Molecular Signatures Database. When the directionality of expression above or below the mean of each gene was mapped, a clear similarity between treated and young cells as opposed to aged cells for both fibroblasts and endothelial cells was observed. Principal component analysis (PCA) in this gene set space was performed and, it was determined that the young and aged populations were separable along the first principal component (PC1), which explained 64.8% of variance in fibroblasts and 60.9% of variance in endothelial cells. Intriguingly, the treated cells also clustered closer to the younger population along PC1 (FIGS. 1K and 2J).

Using the same significance criteria defined above, the untreated and treated aged populations (“UA vs TA”) were compared and found that 1042 genes in fibroblasts (734 upregulated, 308 downregulated) and 992 in endothelial cells (461 upregulated, 531 downregulated) were differentially expressed. Interestingly, also within these sets of genes we found enrichment for aging pathways, within the Molecular Signatures Databases. When the profiles young versus untreated aged (“Y vs UA”) and untreated aged versus treated aged (“UA vs TA”) in each cell type were compared, a 24.7% overlap for fibroblasts (odds ratio of 4.53, p<0.05) and 16.7% overlap for endothelial cells (odds ratio of 3.84, p<0.05) was observed with the directionality of change in gene expression matching that of youth (i.e. if higher in young then higher in treated aged); less than 0.5% moved oppositely in either cell types.

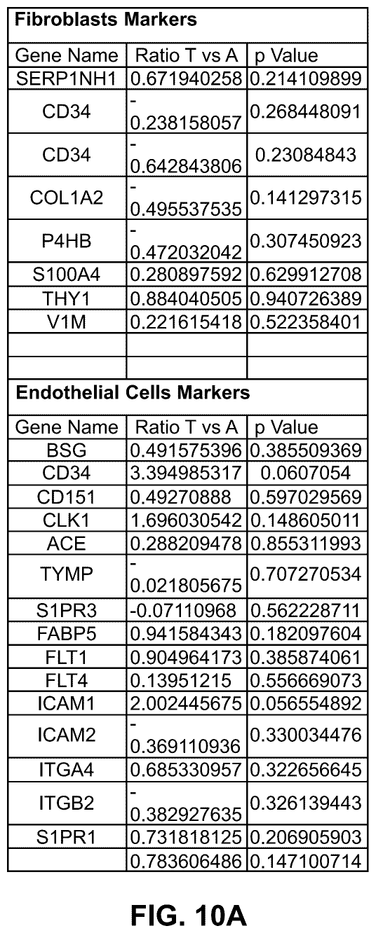

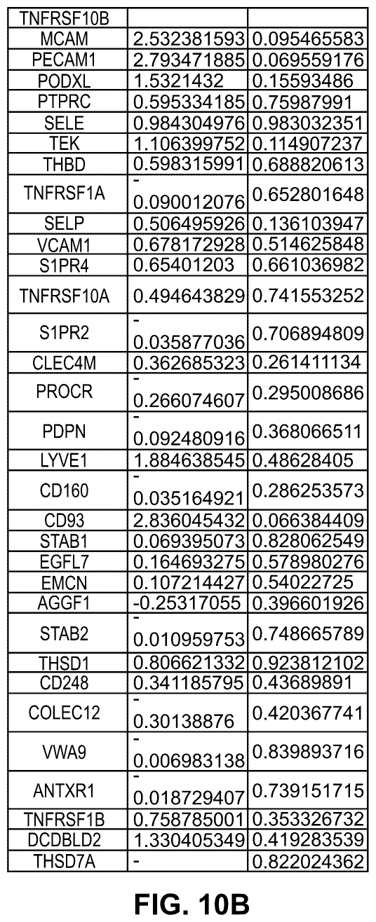

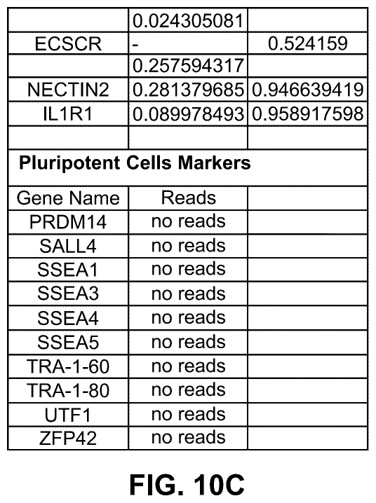

Next, these transcriptomic profiles were used to verify retention of cell identity after transient reprogramming. To this end, using established cell identity markers, we verified that none significantly changed upon treatment (FIG. 10). In addition, we could not detect the expression of any pluripotency-associated markers (other than the OSKMLN mRNAs transfected in) (FIG. 10). Altogether, the analysis of the transcriptomic signatures revealed that transient reprogramming triggers a more youthful gene expression profile, while retaining cell identity.

Epigenetic clocks based on DNA methylation levels are the most accurate molecular biomarkers of age across tissues and cell types and are predictive of a host of age-related conditions including lifespan. Exogenous expression of canonical reprogramming factors (OSKM) is known to revert the epigenetic age of primary cells to a prenatal state. To test whether transient expression of OSKMLN could reverse the epigenetic clock, two epigenetic clocks that apply to human fibroblasts and endothelial cells were used: Horvath's original pan-tissue epigenetic clock (based on 353 cytosine-phosphate-guanine pairs), and skin & blood clock (based on 391 CpGs).

According to the pan tissue epigenetic clock, transient OSKMLN significantly (two-sided mixed effects model P-value=0.023) reverted the DNA methylation age (average age difference=−3.40 years, standard error 1.17). The rejuvenation effect was more pronounced in endothelial cells (average age difference=−4.94 years, SE=1.63, FIG. 6H) than in fibroblasts (average age difference=−1.84, SE=1.46, FIG. 6G). Qualitatively similar, but less significant results, could be obtained with the skin and blood epigenetic clock (overall rejuvenation effect −1.35 years, SE=0.67, one-sided mixed effects model P-value=0.042, average rejuvenation in endothelial cells and fibroblasts is −1.62 years and −1.07, respectively).

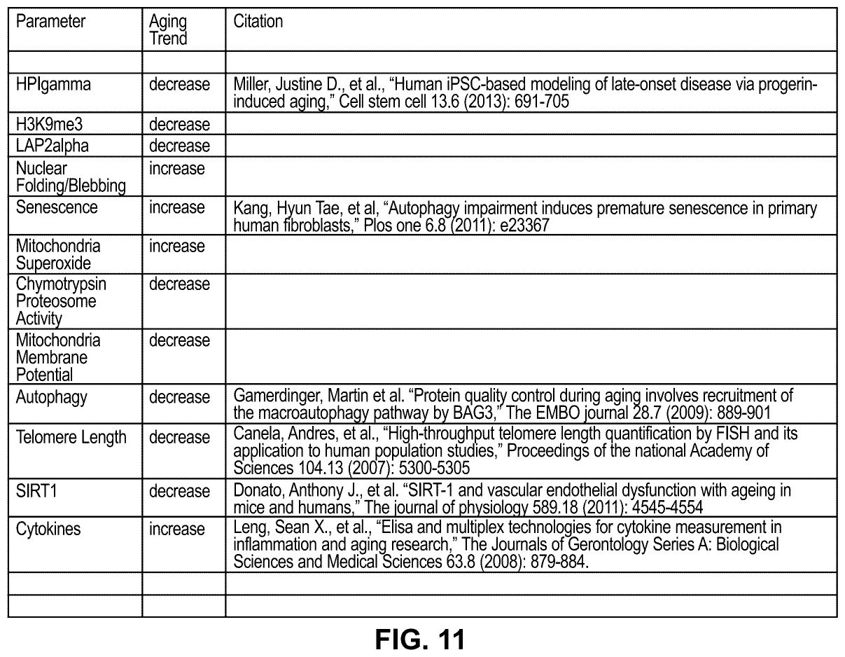

Prompted by these results, the effect of transient reprogramming on various hallmarks of cellular physiological aging was analyzed. A panel of 11 established assays, spanning the hallmarks of aging was employed (FIG. 11), and most of the analyses was performed using single cell high throughput imaging to capture quantitative changes in single cells and distribution shifts in the entire population of cells. All the analyses were performed separately in each individual cell line (total of 19 fibroblast lines: 3 young, 8 aged and 8 treated aged; total 17 endothelial cell lines: 3 young, 7 aged and 7 treated aged). Statistical analysis was conducted in each paired sets of samples; the data was subsequently pooled by age category for ease of representation (see Materials and Methods for a detailed description of the Statistical methods that were used). Control experiments were performed by adopting the same transfection scheme using mRNA encoding for GFP.

To extend the findings on epigenetics, experiments were conducted to quantitatively measure by immunofluorescence (IF) the epigenetic repressive mark H3K9me3, the heterochromatin-associated protein HP1γ, and the nuclear lamina support protein LAP2α (FIGS. 1B, 1C, 2B, 2C, 5A-C) Aged fibroblasts and endothelial cells showed a decrease in the nuclear signal for all three markers compared to young cells. The treatment of aged cells resulted in an increase of these markers in both cell types. Next, both pathways involved in proteolytic activity of the cells were examined by measuring formation of autophagosomes, and chymotrypsin-like proteasomal activity, which decreases with age. Treatment increased both pathways to levels similar to or even higher than young cells, suggesting that early steps in reprogramming promote an active clearance of degraded biomolecules (FIG. 1D, 2D, 5G-H).

In terms of energy metabolism, aged cells display decreased mitochondrial activity, accumulation of reactive oxygen species (ROS), and deregulated nutrient sensing. Therefore, the effects of treatment on aged cells was tested by measuring mitochondria membrane potential, mitochondrial ROS, and levels of Sirtuin1 protein (SIRT1) in the cells. Transient reprogramming increased mitochondria membrane potential in both cell types (FIGS. 1E left panel, 2E left panel, 5E), while it decreased ROS (FIGS. 1E right panel, 2E right panel, 5F) and increased SIRT1 protein levels in fibroblasts, similar to young cells (FIGS. 1F, 2F, and 5D). Senescence-associated beta-galactosidase staining showed a significant reduction in the number of senescent cells in aged endothelial cells (FIGS. 1H, 2H, and 5I). This decrease was accompanied by a decrease in pro-inflammatory senescence associated secretory phenotype (SASP) cytokines (5J) again in endothelial cells. Lastly, in neither cell type did telomere length, measured by quantitative fluorescence in situ hybridization show significant extension with treatment (FIGS. 1G and 2G), suggesting that the cells did not de-differentiate into a stem-like state in which telomerase activity would be reactivated.

Next, the perdurance of these effects was assessed and found that most were significantly retained after four and six days from the interruption of reprogramming. How rapidly these physiological rejuvenative changes manifest was investigated by repeating the same sets of experiments in fibroblasts and endothelial cells that were transfected for just two consecutive days. Remarkably, data showed that most of the rejuvenative effects could already be seen after two days of treatment, although most were more moderate.

Collectively, this data demonstrate that transient expression of OSKMLN can induce a rapid, persistent reversal of cellular age in human cells at the transcriptomic, epigenetic and cellular levels. Importantly, these data demonstrate that the process of “cellular rejuvenation”—that herein named Epigenetic Reprogramming of Aging, or “ERA”—is engaged very early and rapidly in the iPSC reprogramming process. These epigenetic and transcriptional changes occur before any epigenetic reprogramming of cellular identity takes place.

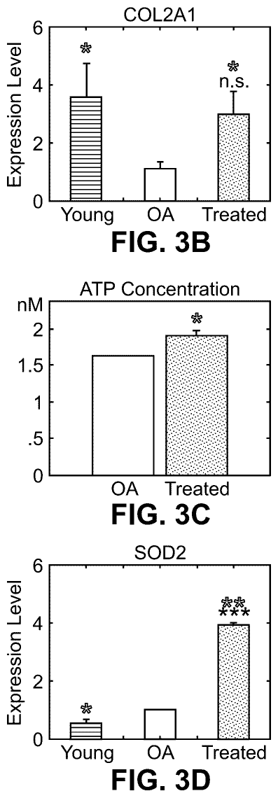

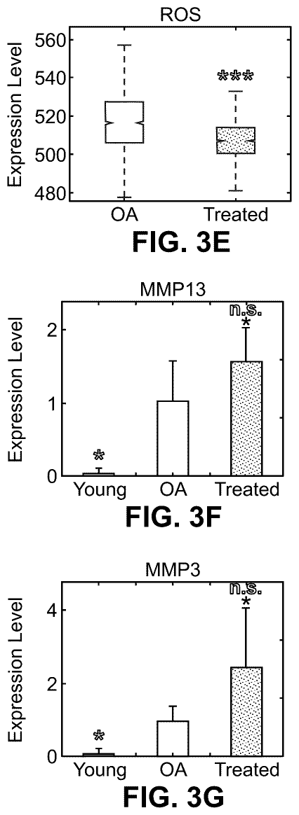

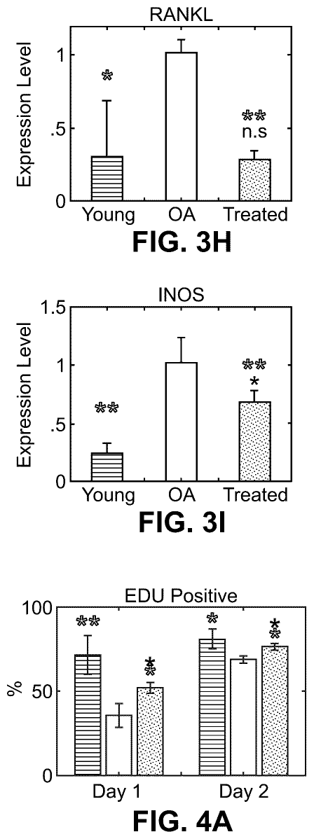

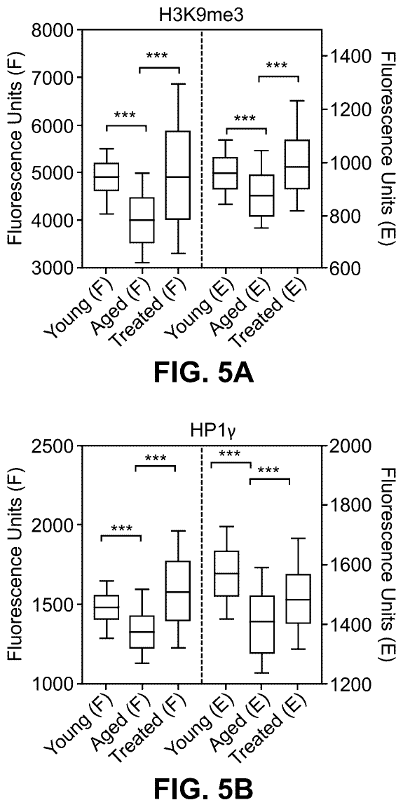

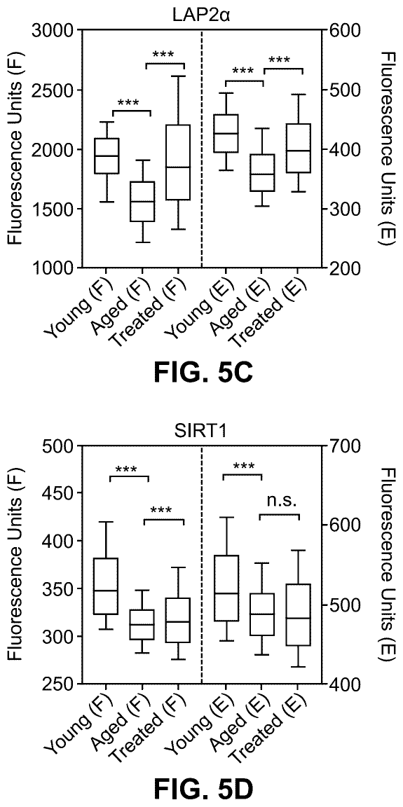

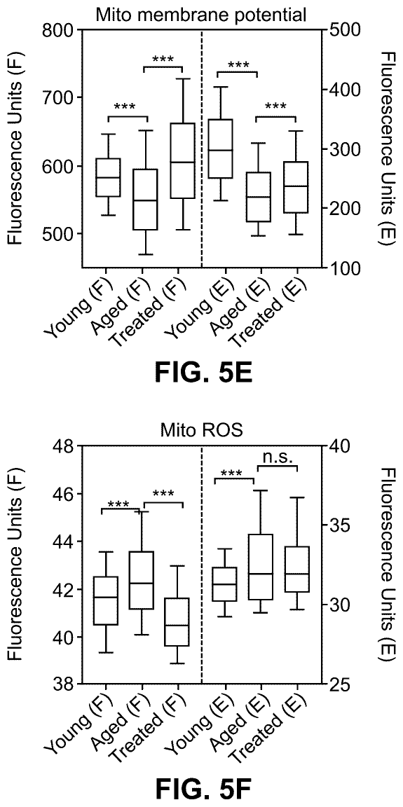

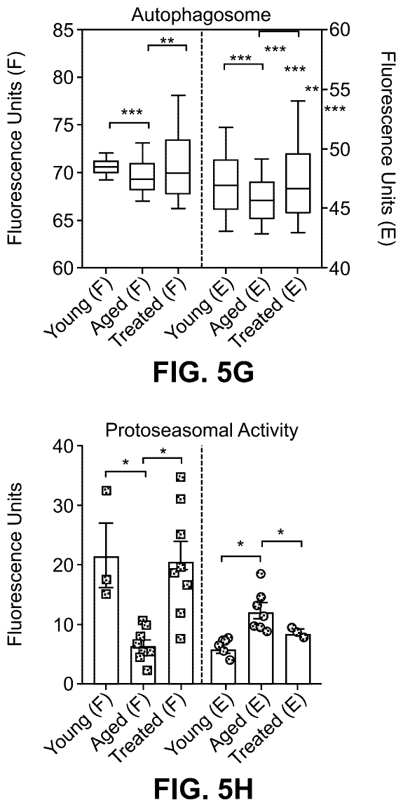

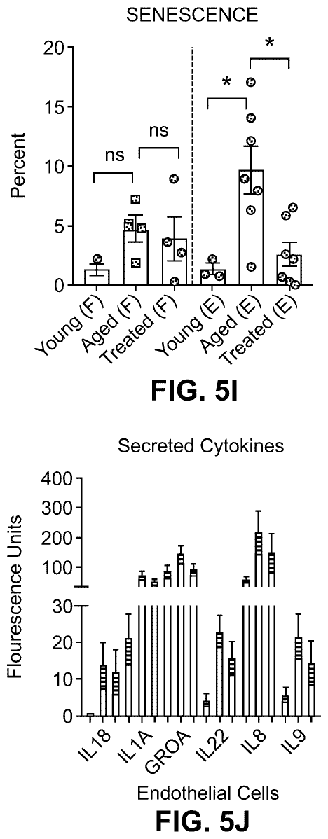

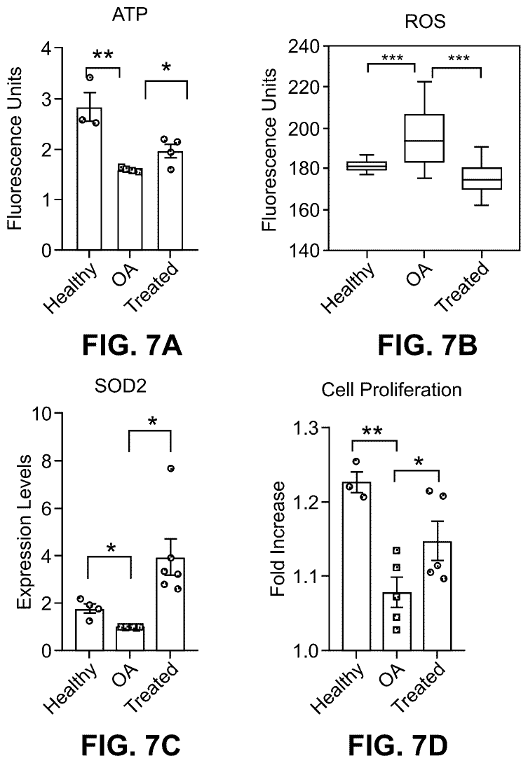

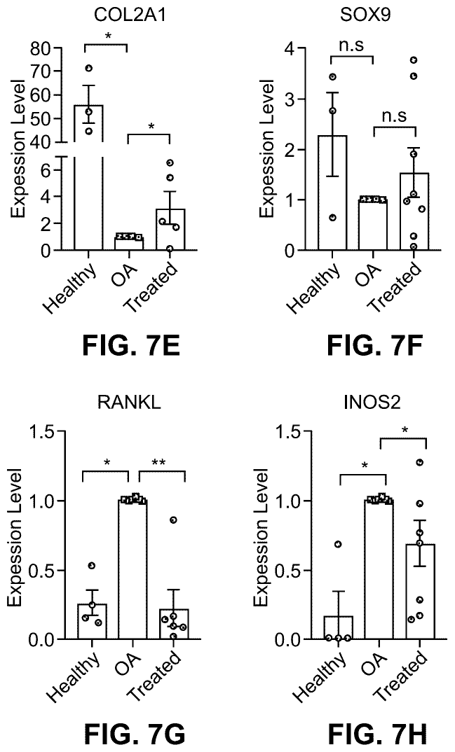

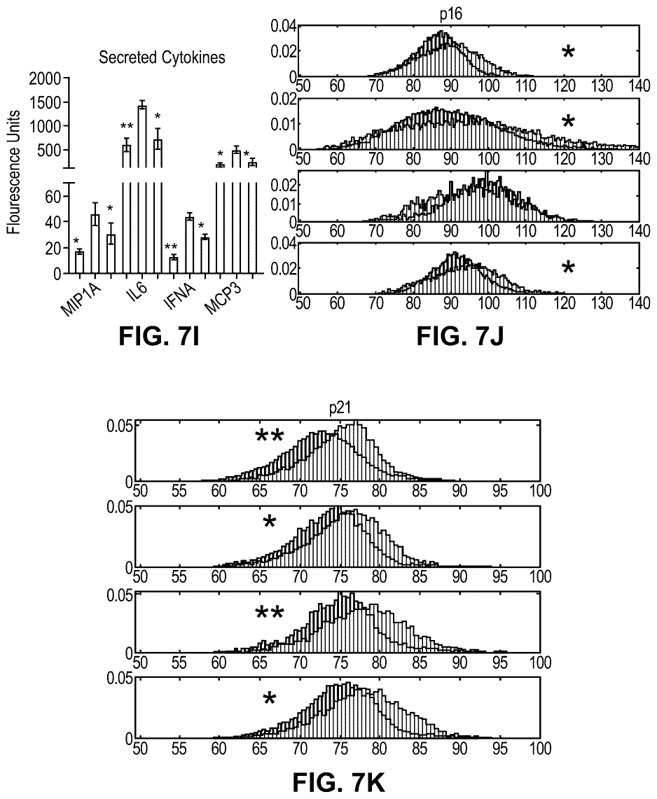

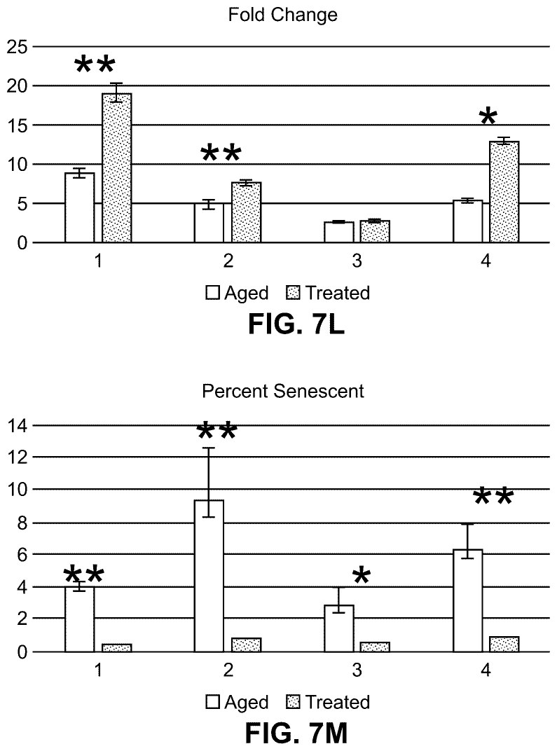

With these indications of a beneficial effect of ERA on cellular aging, experiments were conducted to investigate whether ERA could also reverse the inflammatory phenotypes associated with aging. After obtaining preliminary evidence of this reversal in endothelial cells (FIG. 5J), analysis was extended to osteoarthritis, a disease strongly associated with aging and characterized by a pronounced inflammatory spectrum affecting the chondrocytes within the joint. Chondrocytes were isolated from cartilage of 60-70 year old patients undergoing total joint replacement surgery owing to their advanced stage OA and compared the results of treatment to chondrocytes isolated from young individuals. Transient reprogramming was performed for two or three days and the analysis performed after two days from interruption of reprogramming, though the more consistent effect across patients was with longer treatment. Treatment showed a significant reduction in pro-inflammatory cytokines (FIG. 7I) intracellular mRNA levels of RANKL and iNOS2, as well as in levels of inflammatory factors secreted by the cells (FIGS. 3H-I and 7I). In addition, ERA promoted cell proliferation (FIGS. 3A and 7D), increased ATP production (FIGS. 3C and 7A), and decreased oxidative stress as revealed by reduced mitochondrial ROS and elevated RNA levels of antioxidant SOD2 (FIGS. 3D, 3E, 7B and 7D), a gene that has been shown to be downregulated in OA. ERA did not affect the expression level of SOX9 (a transcription factor core to chondrocyte identity and function) and significantly increased the level of expression of COL2A1 (the primary collagen in articular cartilage) (qRT-PCR in FIGS. 3B, 7E, and 7F), suggesting retention of chrondrogenic cell identity. Together, these results show that transient expression of OSKMLN can promote a partial reversal of gene expression and cellular physiology in aged OA chondrocytes toward a healthier state.

Stem cell loss of function and regenerative capacity represents another important hallmark of aging. Experiments were conducted to assess the effect of transient reprogramming on the age-related changes in somatic stem cells that impair regeneration. First, the effect of transient reprogramming was tested on mouse-derived skeletal muscle stem cells (MuSCs). MuSCs were treated for 2 days while they were kept in a quiescent state using an artificial niche. Initial experiments were conducted with young (3 month) and aged (20-24 months) murine MuSCs isolated by FACS. Treatment of aged MuSCs reduced both time of first division, approaching the faster activation kinetics of quiescent young MuSCs, and mitochondrial mass. Moreover, treatment partially rescued the reduced ability of single MuSCs to form colonies. These cells were further cultured and data showed that treatment did not change expression of the myogenic marker MyoD but instead improved their capacity to differentiate into myotubes, suggesting that transient reprogramming does not disrupt the myogenic fate but can enhance the myogenic potential.

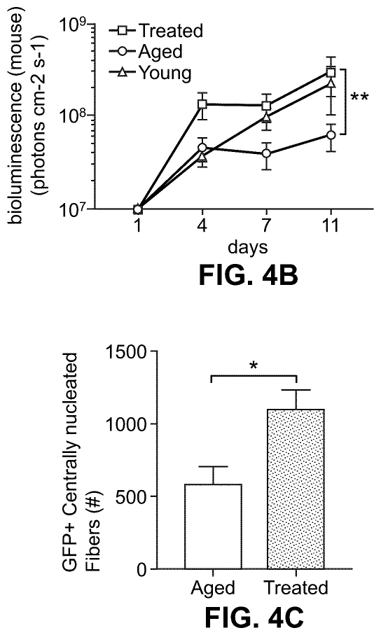

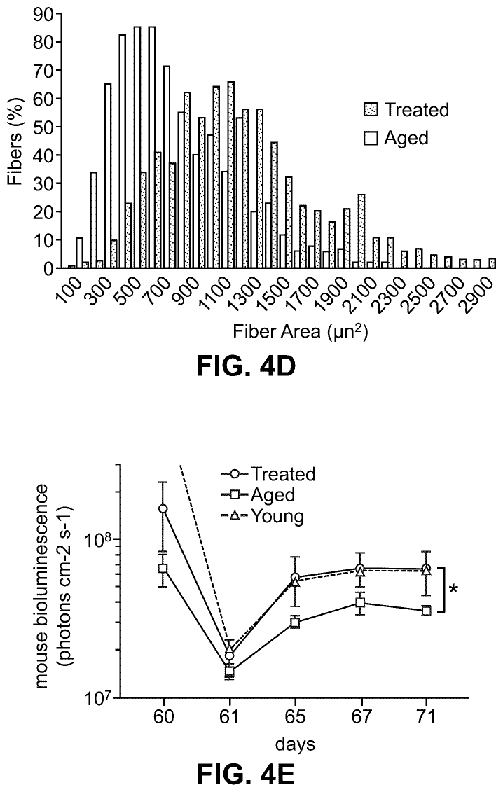

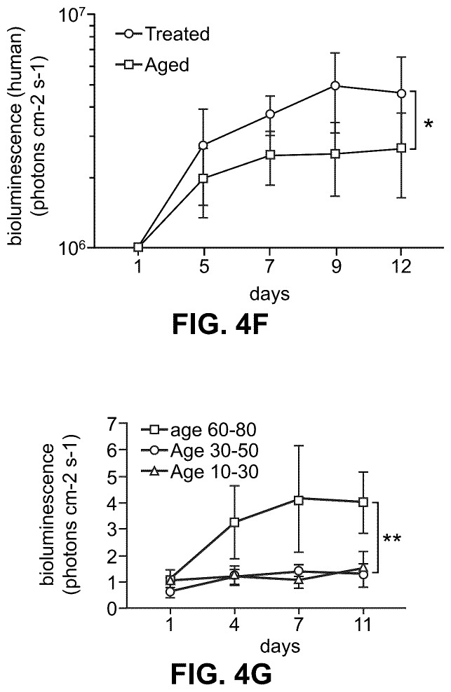

Next, MuSC function and potency to regenerate new tissue in vivo was tested. To do this, young, aged, or transiently reprogrammed aged MuSCs were transduced with a lentivirus expressing luciferase and green fluorescent protein (GFP) and then the cells were transplanted into injured tibialis anterior (TA) muscles of immunocompromised mice. Longitudinal bioluminescence imaging (BLI) initially showed that muscles transplanted with treated aged MuSCs showed the highest signal (day 4, FIG. 4B), but became comparable to muscles with young MuSCs by day 11 post-transplantation; conversely muscle with untreated aged MuSCs showed lower signals at all time points post transplantation (FIG. 4B). Immunofluorescence analysis further revealed higher numbers of donor-derived (GFP⁺) myofibers in TAs transplanted with treated compared to untreated aged MuSCs (FIG. 4C). Moreover, the GFP⁺ myofibers from treated aged cells exhibited increased cross-sectional areas when compared to their untreated counterparts, and in fact even larger than the young controls (FIG. 4D). Together, these results suggest improved tissue regenerative potential of transiently reprogrammed aged MuSCs. After 3 months, all mice were subjected to autopsy, and no neoplastic lesions or teratomas were discovered.

To test potential long-term benefits of the treatment, a second injury was induced 60 days after cell transplantation, and again data showed that TA muscles transplanted with transiently reprogrammed aged MuSCs yielded higher BLI signals (FIG. 4E).

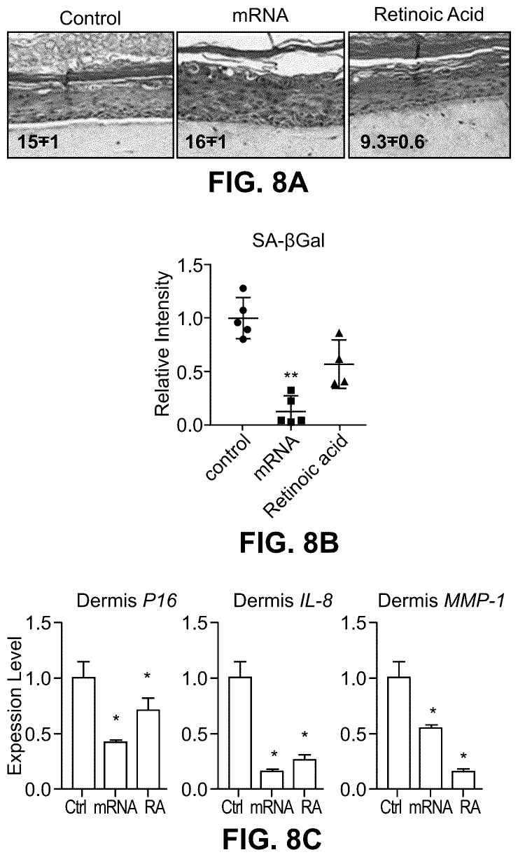

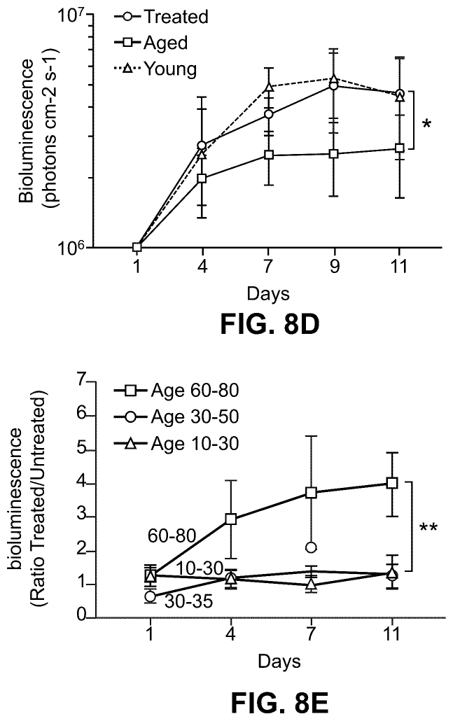

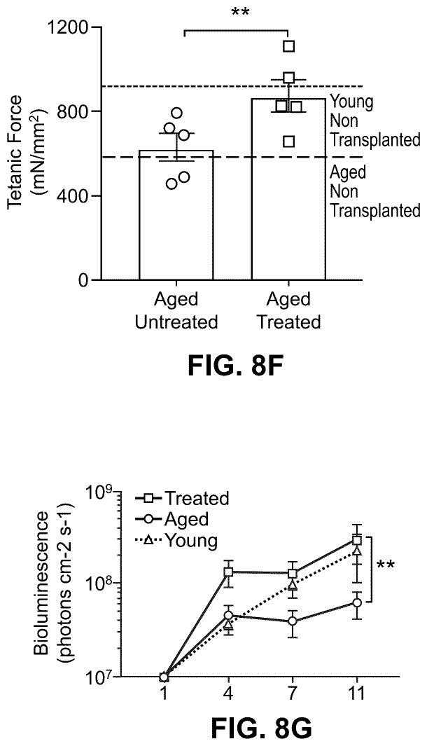

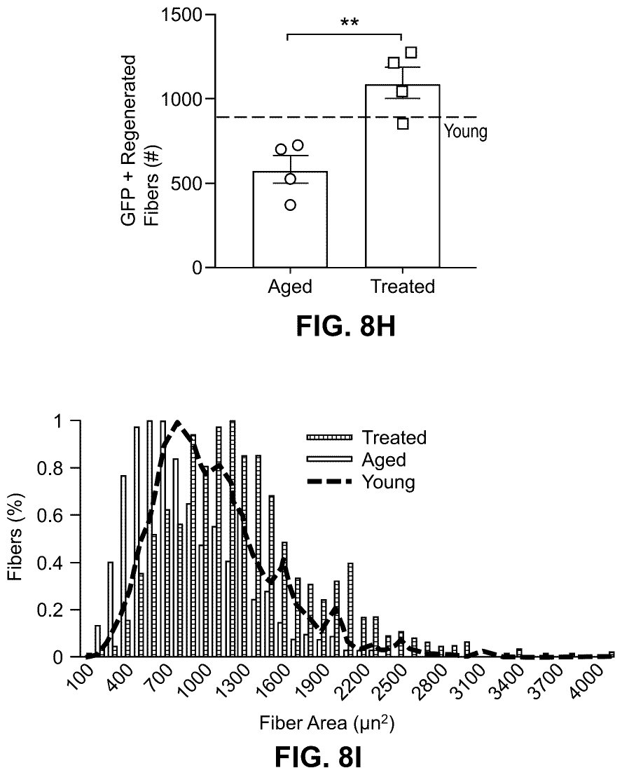

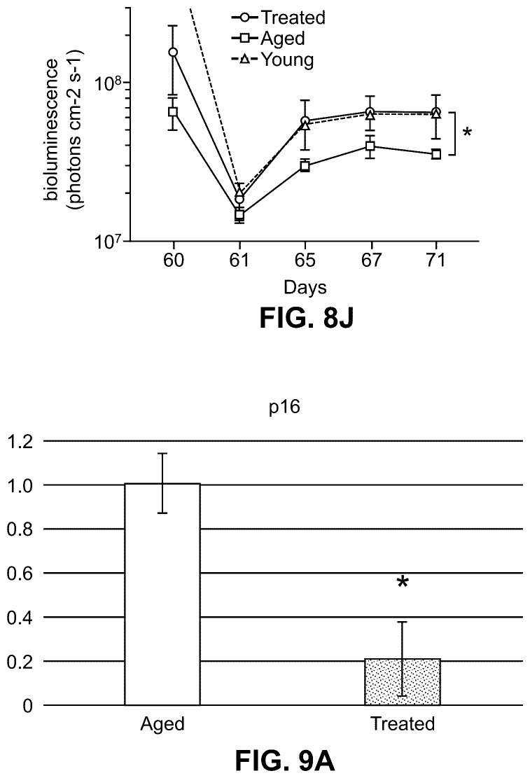

Sarcopenia is an age-related condition that is characterized by loss of muscle mass and force production. Similarly, in mice muscle functions show progressive degeneration with age. To test whether transient reprogramming of aged MuSCs would improve a cell-based treatment in restoring physiological functions of muscle of older mice, electrophysiology was performed to measure tetanic force production in TA muscles isolated from young (4 months) or aged (27 months) immunocompromised mice. Data showed that TA muscles from aged mice have lower tetanic forces compared to young mice, suggesting an age-related loss of force production (FIG. 8F). Next, MuSCs were isolated from aged mice (20-24 months). After treating aged MuSCs, the cells were transplanted into cardiotoxin-injured TA muscles of aged (27 months) immunocompromised mice. Thirty (30) days was provided to give enough time to the transplanted muscles to fully regenerate. Electrophysiology was performed to measure tetanic force production. Muscles transplanted with untreated aged MuSCs showed forces comparable to untransplanted muscles from aged control mice (FIG. 4h). Conversely, muscles that received treated aged MuSCs showed tetanic forces comparable to untransplanted muscles from young control mice. These results support transient reprogramming in combination with MuSC-based therapy can restore physiological function of aged muscles to that of youthful muscles.

Lastly, these results were translated to human MuSCs. The study was repeated, employing operative samples obtained from patients in different age ranges (10 to 80 years old), and transducing them with GFP- and luciferase-expressing lentiviral vectors. As in mice, transplanted, transiently-reprogrammed, aged human MuSCs resulted in increased BLI signals compared to untreated MuSCs from the same individual and comparable to those observed with young MuSCs (FIG. 8D). Interestingly, the BLI signal ratio between contralateral muscles with treated and untreated MuSCs was higher in the older age group (60-80 year old) than in the younger age groups (10-30 or 30-55 years old), suggesting that ERA restores lost functions to younger levels in aged cells (FIG. 8E). Taken together, these results suggest that transient reprogramming partially restores the potency of aged MuSCs to a degree similar to that of young MuSCs, without compromising their fate, and thus has potential as a cell therapy in regenerative medicine.

Three-dimensional (3D) in vitro engineered skins were reconstituted combining fibroblasts and keratinocytes from >65 old patients, and transfected by adding the cocktail of reprogramming factors to the culture media. Histological analysis was performed to assess the quality and a numerical score was assigned (FIG. 8A). Rejuvenation was observed with reprogramming factors as measured by increased numerical score compared to control untreated and retinoic acid treated samples.

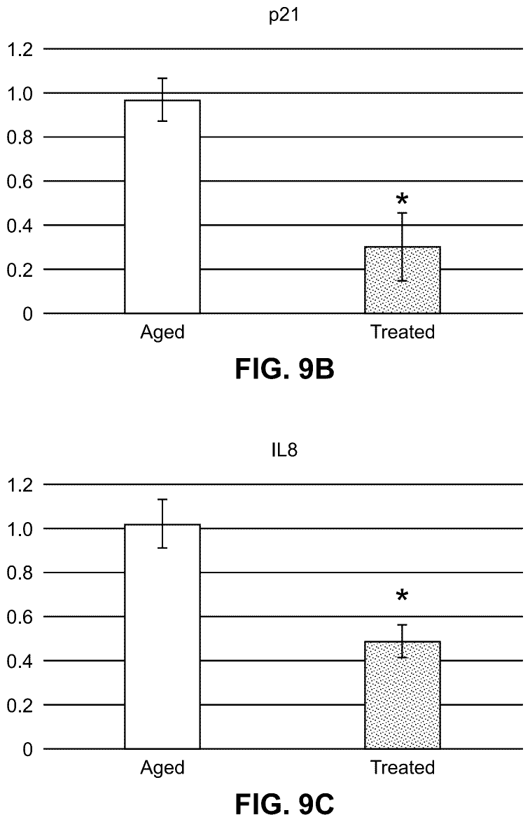

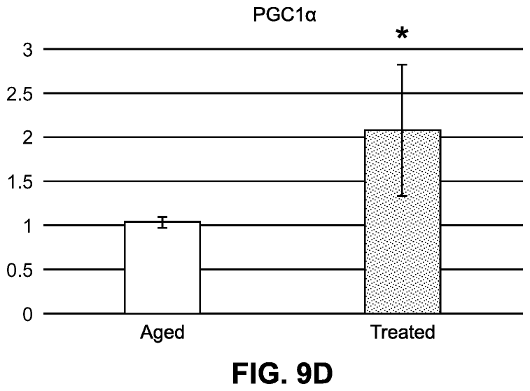

Retinal epithelial cells were cultured ex-vivo and transiently reprogrammed with OSKMN for two or three days. Results showed significant decrease of expression of p16 (FIG. 9A), p21 (FIG. 9B), IL8 and (FIG. 9C) PGC1a (FIG. 9D).

Nuclear reprogramming to induced pluripotent stem cells (iPSCs) is a multi-phased process comprising initiation, maturation and stabilization. Upon completion of such a dynamic and complex “epigenetic reprogramming”, iPSCs are not only pluripotent but also youthful. The data herein demonstrated that a non-integrative, mRNAs-based platform of transient cellular reprogramming can very rapidly reverse hallmarks of aging in the initiation phase, when epigenetic erasure of cell identity has not yet occurred. The data shows that the process of rejuvenation occurs in aged human cells, with restoration of lost functionality in diseased cells and aged stem cells while preserving cellular identity.

### Example 2: Methods

mRNA Transfection: Cells were transfected using either mRNA-In (mTI Global Stem) for Fibroblasts and Chondrocytes, to reduce cell toxicity, and Lipofectamine MessengerMax (Thermo Fisher) for Endothelial Cells and MuSCs, which were more difficult to transfect, using manufacturer's protocol. Culture medium was changed for Fibroblasts and Endothelial Cells 4 hours after transfection, but not for Chondrocytes or MuSCs as overnight incubation was needed to produce a significant uptake of mRNA. Efficiency of delivery was confirmed by both GFP mRNA and immunostaining for individual factors in OSKMNL cocktail. mRNA synthesis and transfection optimization were done together with Jens Durruthy-Durruthy, also a member of the Sebastiano Lab, and the facilities at ESI BIO, for which he was a consultant.

Fibroblast Isolation and Culture: Isolation was performed on healthy patient biopsied from mesial aspect of mid-upper arm or abdomen using 2 mm-punch biopsies from a mix of male and female patients in their 60-70's (aged) and 30-40's (young). Cells were cultured out from these explants and maintained in Eagle's, Minimum Essential Medium with Earl's salts supplemented with non-essential amino acids, 10% fetal bovine serum and 1% Penicillin/Streptomycin.

Endothelial Cell Isolation and Culture: Isolation was performed at Coriell Institute from iliac artery and vein removed ante-mortem from donors that died of sudden head trauma but were otherwise healthy in their 45-50's (aged) and teens (young). Tissue was digested with collagenase and cells released from the lumen were used to initiate culture. Cells were maintained in Medium 199 supplemented with 2 mM L-glutamine, 15 fetal bovine serum, 0.02 mg/ml Endothelial Growth Supplement, 0.05 mg/ml Heparin and 1% Penicillin/Streptomycin.

Nuclear Immunocytochemistry: Cells were washed with HBSS then fixed with 15% Paraformaldehyde in PBS for 15 minutes. Cells were then blocked for 30 minutes with a blocking solution of 1% BSA and 0.3% Triton X-100 in PBS. Primary antibodies were then applied in 1% BSA and 0.3% Triton X-100 in PBS and allowed to incubate overnight at 4C. The following day the cells were wash with HBSS before switching to the corresponding Alexa Flour-labeled secondary antibodies and incubated for 2 hours. The cells were then washed again and then stained with DAPI for 30 minutes. Finally, the cells were switched to HBSS for imaging.

Autophagosome Formation Staining: Cells were washed with HBSS and switched to a staining solution containing a LC3 based fluorescent autophagosome marker (Sigma). The cells were then incubated at 37° C. with 5% CO2 for 20 minutes. Cells were then washed 2 times using HBSS/Ca/Mg. Cells were then stained for 15 minutes using CellTracker Deep Red, cell labeling dye. Cells were then switched to HBSS/Ca/Mg for single cell imaging with Operetta

Proteasome Activity Measurement: Wells were first stained with PrestoBlue (Thermo), cell viability dye, for 10 minutes. Well signals were read using TECAN fluorescent plate reader. Then cells were washed with HBSS/Ca/Mg before switching to original media containing LLVY-R110 fluorogenic substrate (Sigma) which is cleaved by chymotrypsin like 20S proteasome activity. Cells were then incubated at 37° C. with 5% CO2 for 2 hours, before reading again on the TECAN fluorescent plate reader.

Mitochondria Membrane Potential Staining: Tetramethylrhodamine, Methyl Ester, Perchlorate (Thermo) was added to cell culture media, this dye is sequestered by mitochondria based on their membrane potential. Cells were then incubated for 30 minutes at 37° C. with 5% CO2. Cells were then washed 2 times with HBSS/Ca/Mg before staining for 15 minutes using CellTracker Deep Red. Finally, cells were imaged in fresh HBSS/Ca/Mg with Operetta

Mitochondria ROS Measurement: Cells were washed with HBSS/Ca/Mg then switch to HBSS/Ca/Mg containing MitoSOX, a fluorogenic dye that is oxidized by superoxides in the mitochondria. Cells were the incubated for 10 minutes at 37 C with 5% CO2. Cells were then washed twice with HBSS/Ca/Mg, then stained for 15 minutes using CellTracker Deep Red. Finally, cells were imaged in fresh HBSS/Ca/Mg with Operetta

SaβGal Histochemistry: Cells were washed twice with HBSS/Ca/Mg then fixed with 15% Paraformaldehyde in PBS for 6 minutes. Cells were then rinsed 3 times with HBSS/Ca/Mg before staining with X-gal chromogenic substrate, cleaved by endogenous B galactosidase. Cells were kept in the staining solution and incubated overnight at 37° C. with ambient CO2. The next day, cells were washed again HBSS/Ca/Mg before switching to a 70% glycerol solution for imaging under a Leica brightfield microscope

Cytokine Profiling: This work was performed together with the Human Immune Monitoring Center at Stanford University. Cell media was harvested and spun at 400 rcf for 10 minutes at RT. The supernatant was then snap frozen with liquid nitrogen until analysis. Analysis was done using the human 63-plex kit (eBiosciences/Affymetrix). Beads were added to a 96 well plate and washed in a Biotek ELx405 washer. Samples were added to the plate containing the mixed antibody-linked beads and incubated at room temperature for 1 hour followed by overnight incubation at 4° C. with shaking. Cold and Room temperature incubation steps were performed on an orbital shaker at 500-600 rpm. Following the overnight incubation plates were washed in a Biotek ELx405 washer and then biotinylated detection antibody added for 75 minutes at room temperature with shaking. Plate was washed as above and streptavidin-PE was added. After incubation for 30 minutes at room temperature wash was performed as above and reading buffer was added to the wells. Each sample was measured in duplicate. Plates were read using a Luminex 200 instrument with a lower bound of 50 beads per sample per cytokine. Custom assay Control beads by Radix Biosolutions are added to all wells.

Antibodies: 5 primary antibodies were used for nuclear measurements: Rabbit Anti-Histone H3K9me3 histone methylation (1:4000), Mouse Anti-HP1γ heterochromatin marker (1:200), Rabbit Anti-LAP2α (1:500) nuclear organization protein, Mouse Anti-LAMININ A/C nuclear envelope marker and Rabbit Anti-SIRT1 (1:200).

### RNA-Sequencing and Data Analysis

Cells were washed and digested by TRIzol (Thermo). Total RNA was isolated using the Total RNA Purification Kit (Norgen Biotek Corp) and RNA quality was assessed by the RNA analysis screentape (R6K screentape, Agilent), RNA with RIN>9 was reverse transcribed to cDNA. The cDNA libraries were prepared using 1 μg of total RNA using the TruSeq RNA Sample Preparation Kit v2 (Illumina). RNA quality was assessed by the Agilent Bioanalyzer 2100, RNA with RIN>9 was reverse transcribed to cDNA. The cDNA libraries were prepared using 500 ng of total RNA using the TruSeq RNA Sample Preparation Kit v2 (Illumina) with the added benefit of molecular indexing. Prior to any PCR amplification steps, all cDNA fragment ends were ligated at random to a pair of adapters containing an 8 bp unique molecular index. The molecular indexed cDNA libraries were than PCR amplified (15 cycles) and then QC'ed using a Bioanalzyer and Qubit. Upon successful QC, they were sequenced on the Illumina Nextseq platform to obtain 80-bp single-end reads. The reads were trimmed, 2 nucleotides on each end, to remove low quality parts, and improve mapping to the genome. The 78 nucleotide reads that resulted were compressed by removing duplicates, but keeping track of how many times each sequence occurred in each sample in a database. The unique reads were then mapped to the human genome, using exact matches. This misreads that cross exon-exon boundaries, as well as reads with errors and SNPs/mutations, but it does not have substantial impact on estimating the levels of expression of each gene. Each mapped read was then assigned annotations from the underlying genome. In case of multiple annotations (e.g. a miRNA occurring in the intron of a gene), a hierarchy based on heuristics was used to give a unique identity to each read. This was then used to identify the reads belonging to each transcript and coverage over each position on the transcript was established. This coverage is non-uniform and spiky, thus we used the median of this coverage as an estimate of the gene's expression value. In order to compare the expression in different samples, quantile normalization was used. Further data analysis was done in MATLAB. Ratios of expression levels were then calculated to estimate the log (to base 2) of the fold-change. Student's t-test was used to determine significance with a p<0.05 cutoff. ENCODE gene analysis was used for transcription factor identification, which was developed and made publicly availably the by the Butte Lab in the Stanford Center for Biomedical Informatics Research.

Mice: C57BL/6 male mice and NSG mice were obtained from Jackson Laboratory. NOD/MrkBomTac-Prkdcscid female mice were obtained from Taconic Biosciences. Mice were housed and maintained in the Veterinary Medical Unit at the Veterans Affairs Palo Alto Health Care Systems. Animal protocols were approved by the Administrative Panel on Laboratory Animal Care of Stanford University.

Human skeletal muscle specimens: Subjects ranged in age from 10 to 78 years. The human muscle biopsy specimens were taken after patients gave informed consent, as part of a human studies research protocol which was approved by the Stanford University Institutional Review Board. All experiments were performed using fresh muscle specimens, according to availability of the clinical procedures. Sample processing for cell analysis began within one to twelve hours of specimen isolation. In all studies, standard deviation reflects variability in data derived from studies using true biological replicates (that is, unique donors). Data were not correlated with donor identity.

MuSC Isolation and Purification: Muscles were harvested from hind limbs and mechanically dissociated to yield a fragmented muscle suspension. This was followed by a 45-50 minute digestion in a Collagenase II-Ham's F10 solution (500 units per ml; Invitrogen). After washing, a second digestion was performed for 30 minutes with Collagenase II (100 units per ml) and Dispase (2 units per ml; ThermoFisher). The resulting cell suspension was washed, filtered and stained with VCAM-biotin (clone 429; BD Bioscience), CD31-FITC (clone MEC 13.3; BD Bioscience), CD45-APC (clone 30-F11; BD Bioscience) and Sca-1-Pacific-Blue (clone D7; Biolegend) antibodies at a dilution of 1:100. Human MuSCs were purified from fresh operative samples50,51. Operative samples were carefully dissected from adipose and fibrotic tissue and a disassociated muscle suspension prepared as described for mouse tissue. The resulting cell suspension was then washed, filtered and stained with anti-CD31-Alexa Fluor 488 (clone WM59; BioLegend; #303110, 1:75), anti-CD45-Alexa Fluor 488 (clone HI30; Invitrogen; #MHCD4520, 1:75), anti-CD34-FITC (clone 581; BioLegend; #343503, 1:75), anti-CD29-APC (clone TS2/16; BioLegend; #303008, 1:75) and anti-NCAM-Biotin (clone HCD56; BioLegend; #318319, 1:75). Unbound primary antibodies were then washed and the cells incubated for 15 min at 4° C. in streptavidin-PE/Cy7 (BioLegend) to detect NCAM-biotin. Cell sorting was performed on calibrated BD-FACS Aria II or BD FACSAria III flow cytometers equipped with 488-nm, 633-nm and 405-nm lasers to obtain the MuSC population. A small fraction of sorted cells was plated and stained for Pax7 and MyoD to assess the purity of the sorted population. For the FACS gating strategy, see Supplementary Information.

Bioluminescence Imaging: Bioluminescent imaging was performed using the Xenogen IVIS-Spectrum System (Caliper Life Sciences). Mice were anesthetized using 2% isoflurane at a flow rate of 2.5 l/min (n=4). Intraperitoneal injection of D-Luciferin (50 mg/ml, Biosynth International Inc.) dissolved in sterile PBS was administered. Immediately following the injection, mice were imaged for 30 seconds at maximum sensitivity (f-stop 1) at the highest resolution (small binning). Every minute a 30 second exposure was taken, until the peak intensity of the bioluminescent signal began to diminish. Each image was saved for subsequent analysis. Imaging was performed in bind: the investigators performing the imaging did not know the identity of the experimental conditions for the transplanted cells.

Bioluminescence Image Analysis: Analysis of each image was performed using Living Image Software, version 4.0 (Caliper Life Sciences). A manually-generated circle was placed on top of the region of interest and resized to completely surround the limb or the specified region on the recipient mouse. Similarly, a background region of interest was placed on a region of a mouse outside the transplanted leg.

Tissue Harvesting: TA muscles were carefully dissected away from the bone, weighed, and placed into a 0.5% PFA solution for fixation overnight. The muscles were then moved to a 20% sucrose solution for 3 hours or until muscles reached their saturation point and began to sink. The tissues were then embedded and frozen in Optimal Cutting Temperature (OCT) medium and stored at −80° C. until sectioning. Sectioning was performed on a Leica CM3050S cryostat that was set to generate 10 μm sections. Sections were mounted on Fisherbrand Colorfrost slides. These slides were stored at −20° C. until immunohistochemistry could be performed.

Histology: TA muscles were fixed for 5 hours using 0.5% electron-microscopy-grade paraformaldehyde and subsequently transferred to 20% sucrose overnight. Muscles were then frozen in OCT, cryosectioned at a thickness of 10 μm and stained. For colorimetric staining with Hematoxylin and Eosin (Sigma) or Gomorri Trichrome (Richard-Allan Scientific) samples were processed according to the manufacturer's recommended protocols.

MuSC Immunostaining: A one-hour blocking step with 20% donkey serum/0.3% Triton in PBS was used to prevent unwanted primary antibody binding for all samples. Primary antibodies were applied and allowed to incubate over night at 4° C. in 20% donkey serum/0.3% Triton in PBS. After 4 washes with 0.3% PBST, fluorescently-conjugated secondary antibodies were added and incubated at room temperature for 1 hour in 0.3% PBST. After 3 additional rinses each slide was mounted using Fluoview mounting media.

Antibodies: The following antibodies were used in this study. The source of each antibody is indicated. Mouse: GFP (Invitrogen, #A11122, 1:250); Luciferase (Sigma-Aldrich, #L0159, 1:200); Collagen I (Cedarlane Labs, #CL50151AP, 1:200); HSP47 (Abcam, #ab77609, 1:200).

Imaging: Samples were imaged using standard fluorescent microscopy and either a 10× or 20× air objective. Volocity imaging software was used to adjust excitation and emission filters and came with pre-programmed AlexaFluor filter settings which were used whenever possible. All exposure times were optimized during the first round of imaging and then kept constant through all subsequent imaging.

Image Analysis: Image J was used to calculate the percentage of area composed of Collagen by using the color threshold plugin to create a mask of only the area positive for Collagen. That area was then divided over the total area of the sample which was found using the free draw tool. All other analyses were performed using Volocity software and manually counting fibers using the free draw tool and also counting the number of nuclei, eMHC+ fibers, neuromuscular junctions and blood vessels by hand.

Lentiviral Transduction: Luciferase and GFP protein reporters were subcloned into a third generation HIV-1 lentiviral vector (CD51X DPS, SystemBio). To transduce freshly isolated MuSCs cells were plated at a density of 30,000-40,000 cells per well on a Poly-D-Lysine (Millipore Sigma, A-003-E) and ECM coated 8-well chamber slide (Millipore Sigma, PEZGS0896) and were incubated with 5 μl of concentrated virus per well and 8 μg/mL polybrene (Santa Cruz Biotechnology, sc-134220). Plates were spun for 5 minutes at 3200 g, and for 1 hour at 2500 g at 25° C. Cells were then washed with fresh media two times, scraped from plates, and resuspended in the final volume according to the experimental conditions.

MitoTracker staining and flow cytometry analysis: MuSCs undergoing reprogramming and controls were washed twice with pure HamsF10 (no serum or pen/strep). Subsequently, MuSCs were stained with 0.5 μM MitoTracker Green FM (ThermoFisher, M7514) and DAPI for 30 minutes at 37° C., washed three times with pure HamsF10, and analyzed using a BD FACSAria III flow cytometer.

Statistical Analysis: Unless otherwise noted, all statistical analyses were performed using MATLAB R2017a (MathWorks Software) or GraphPad Prism 5 (GraphPad Software). For statistical analysis, t-tests were used. All error bars represent s.e.m.; *p<0.05; **p<0.001; ***p<0.0001.

While the preferred embodiments of the disclosure have been illustrated and described, it will be appreciated that various changes can be made therein without departing from the spirit and scope of the disclosure.

### REFERENCES

1 Zhang, R., Chen, H. Z. & Liu, D. P. The Four Layers of Aging. Cell Syst 1, 180-186, doi:10.1016/j.cels.2015.09.002 (2015).
2 Mahmoudi, S. & Brunet, A. Aging and reprogramming: a two-way street. Curr Opin Cell Biol 24, 744-756, doi:10.1016/j.ceb.2012.10.004 (2012).
3 Booth, L. N. & Brunet, A. The Aging Epigenome. Mol Cell 62, 728-744, doi:10.1016/j.molcel.2016.05.013 (2016).
4 Studer, L., Vera, E. & Cornacchia, D. Programming and Reprogramming Cellular Age in the Era of Induced Pluripotency. Cell Stem Cell 16, 591-600, doi:10.1016/j.stem.2015.05.004 (2015).
5 Horvath, S. DNA methylation age of human tissues and cell types. Genome Biol 14, R115, doi:10.1186/gb-2013-14-10-r115 (2013).
6 Ocampo, A. et al. In Vivo Amelioration of Age-Associated Hallmarks by Partial Reprogramming. Cell 167, 1719-1733 e1712, doi:10.1016/j.cell.2016.11.052 (2016).
7 Miller, J. & Studer, L. Aging in iPS cells. Aging (Albany NY) 6, 246-247, doi:10.18632/aging.100653 (2014).
8 Miller, J. D. et al. Human iPSC-based modeling of late-onset disease via progerin-induced aging. Cell Stem Cell 13, 691-705, doi:10.1016/j.stem.2013.11.006 (2013).
9 Mertens, J. et al. Directly Reprogrammed Human Neurons Retain Aging-Associated Transcriptomic Signatures and Reveal Age-Related Nucleocytoplasmic Defects. Cell Stem Cell 17, 705-718, doi:10.1016/j.stem.2015.09.001 (2015).
10 Durruthy, J. D. & Sebastiano, V. Derivation of GMP-compliant integration-free hiPSCs using modified mRNAs. Methods Mol Biol 1283, 31-42, doi:10.1007/7651_2014_124 (2015).
11 Durruthy-Durruthy, J. et al. The primate-specific noncoding RNA HPAT5 regulates pluripotency during human preimplantation development and nuclear reprogramming. Nature genetics 48, 44-52, doi:10.1038/ng.3449 (2016).
12 Liberzon, A. et al. The Molecular Signatures Database (MSigDB) hallmark gene set collection. Cell Syst 1, 417-425, doi:10.1016/j.cels.2015.12.004 (2015).
13 Marioni, R. E. et al. DNA methylation age of blood predicts all-cause mortality in later life. Genome Biol 16, 25, doi:10.1186/s13059-015-0584-6 (2015).
14 Horvath, S. & Raj, K. DNA methylation-based biomarkers and the epigenetic clock theory of ageing. Nat Rev Genet 19, 371-384, doi:10.1038/s41576-018-0004-3 (2018).
15 Chen, B. H. et al. DNA methylation-based measures of biological age: meta-analysis predicting time to death. Aging (Albany NY) 8, 1844-1865, doi:10.18632/aging.101020 (2016).
16 Horvath, S. et al. Epigenetic clock for skin and blood cells applied to Hutchinson Gilford Progeria Syndrome and ex vivo studies. Aging (Albany NY) 10, 1758-1775, doi:10.18632/aging.101508 (2018).
17 Lopez-Otin, C., Blasco, M. A., Partridge, L., Serrano, M. & Kroemer, G. The hallmarks of aging. Cell 153, 1194-1217, doi:10.1016/j.cell.2013.05.039 (2013).
18 Gamerdinger, M. et al. Protein quality control during aging involves recruitment of the macroautophagy pathway by BAG3. EMBO J 28, 889-901, doi:10.1038/emboj.2009.29 (2009).
19 Kang, H. T., Lee, K. B., Kim, S. Y., Choi, H. R. & Park, S. C. Autophagy impairment induces premature senescence in primary human fibroblasts. PloS one 6, e23367, doi:10.1371/journal.pone.0023367 (2011).
20 Donato, A. J. et al. SIRT-1 and vascular endothelial dysfunction with ageing in mice and humans. J Physiol 589, 4545-4554, doi:10.1113/jphysiol.2011.211219 (2011).
21 Hamilton, J. A. GM-CSF in inflammation and autoimmunity. Trends Immunol 23, 403-408 (2002).
22 Gerdes, N. et al. Expression of interleukin (IL)-18 and functional IL-18 receptor on human vascular endothelial cells, smooth muscle cells, and macrophages: implications for atherogenesis. J Exp Med 195, 245-257 (2002).
23 Canela, A., Vera, E., Klatt, P. & Blasco, M. A. High-throughput telomere length quantification by FISH and its application to human population studies. Proc Natl Acad Sci USA 104, 5300-5305, doi:10.1073/pnas.0609367104 (2007).
24 Zhang, Y. & Jordan, J. M. Epidemiology of osteoarthritis. Clin Geriatr Med 26, 355-369, doi:10.1016/j.cger.2010.03.001 (2010).
25 Scott, J. L. et al. Superoxide dismutase downregulation in osteoarthritis progression and end-stage disease. Ann Rheum Dis 69, 1502-1510, doi:10.1136/ard.2009.119966 (2010).
26 Quarta, M. et al. An artificial niche preserves the quiescence of muscle stem cells and enhances their therapeutic efficacy. Nat Biotechnol 34, 752-759, doi:10.1038/nbt.3576 (2016).
27 Schultz, E. & Lipton, B. H. Skeletal muscle satellite cells: changes in proliferation potential as a function of age. Mech Ageing Dev 20, 377-383 (1982).
28 Collins-Hooper, H. et al. Age-related changes in speed and mechanism of adult skeletal muscle stem cell migration. Stem Cells 30, 1182-1195, doi:10.1002/stem.1088 (2012).
29 Garcia-Prat, L. et al. Autophagy maintains stemness by preventing senescence. Nature 529, 37-42, doi:10.1038/nature16187 (2016).
30 Chang, J. et al. Clearance of senescent cells by ABT263 rejuvenates aged hematopoietic stem cells in mice. Nat Med 22, 78-83, doi:10.1038/nm.4010 (2016).
31 Calvani, R. et al. Biomarkers for physical frailty and sarcopenia. Aging Clin Exp Res 29, 29-34, doi: 10.1007/s40520-016-0708-1 (2017).
32 Naranjo, J. D., Dziki, J. L. & Badylak, S. F. Regenerative Medicine Approaches for Age-Related Muscle Loss and Sarcopenia: A Mini-Review. Gerontology 63, 580-589, doi:10.1159/000479278 (2017).
33 Ballak, S. B., Degens, H., de Haan, A. & Jaspers, R. T. Aging related changes in determinants of muscle force generating capacity: a comparison of muscle aging in men and male rodents. Ageing Res Rev 14, 43-55, doi:10.1016/j.arr.2014.01.005 (2014).
34 Sayed, R. K. et al. Identification of morphological markers of sarcopenia at early stage of aging in skeletal muscle of mice. Exp Gerontol 83, 22-30, doi:10.1016/j.exger.2016.07.007 (2016).
35 David, L. & Polo, J. M. Phases of reprogramming. Stem Cell Res 12, 754-761, doi:10.1016/j.scr.2014.03.007 (2014).
36 Yener Ilce, B., Cagin, U. & Yilmazer, A. Cellular reprogramming: A new way to understand aging mechanisms. Wiley Interdiscip Rev Dev Biol 7, doi:10.1002/wdev.308 (2018).
37 Rando, T. A. & Chang, H. Y. Aging, rejuvenation, and epigenetic reprogramming: resetting the aging clock. Cell 148, 46-57, doi:10.1016/j.cell.2012.01.003 (2012).

### P EMBODIMENTS

Embodiment P1. A method of rejuvenating cells, the method including: a) transfecting the cells with one or more non-integrative messenger RNAs encoding one or more cellular reprogramming factors, wherein the transfecting is performed once daily for at least two days and not more than 4 days; and b) translating the one or more non-integrative messenger RNAs to produce the one or more cellular reprogramming factors in the cells resulting in transient reprogramming of the cells, wherein the cells are rejuvenated without dedifferentiation into stem cells.

Embodiment P2. The method of embodiment P1, wherein the one or more cellular reprogramming factors are selected from the group consisting of OCT4, SOX2, KLF4, c-MYC, LIN28 and NANOG.

Embodiment P3. The method of embodiment P2, wherein the cellular reprogramming factors include OCT4, SOX2, KLF4, c-MYC, LIN28 and NANOG.

Embodiment P4. The method of embodiment P1, wherein the cells are mammalian cells.

Embodiment P5. The method of embodiment P4, wherein the cells are human cells.

Embodiment P6. The method of embodiment P1, wherein the cells are from an elderly subject.

Embodiment P7. The method of embodiment P1, wherein the cells are fibroblasts, endothelial cells, chondrocytes, or skeletal muscle stem cells.

Embodiment P8. The method of embodiment P1, wherein the transient reprogramming results in increased expression of HP1gamma, H3K9me3, lamina support protein LAP2alpha, and SIRT1 protein, decreased nuclear folding, decreased blebbing, increased cell autophagosome formation, increased chymotrypsin-like proteasome activity, increased mitochondria membrane potential, or decreased reactive oxygen species (ROS).

Embodiment P9. The method of embodiment P1, wherein the cells are within a tissue or organ.

Embodiment P10. The method of embodiment P9, wherein transient reprogramming reduces numbers of senescent cells within the tissue or organ.

Embodiment P11. The method of embodiment P9, wherein transient reprogramming decreases expression of GMSCF, IL18, and TNFα.

Embodiment P12. The method of embodiment P9, wherein treatment restores function, increases potency, enhances viability, or increases replicative capacity or life span of the cells within the tissue or organ.

Embodiment P13. The method of embodiment P1, wherein the method is performed in vitro, ex vivo, or in vivo.

Embodiment P14. The method of embodiment P1, wherein the transfecting is performed once daily for 3 days or 4 days.

Embodiment P15. A method for treating a subject for an age-related disease or condition, the method including: a) transfecting cells in need of rejuvenation in vivo or ex vivo with one or more non-integrative messenger RNAs encoding one or more cellular reprogramming factors, wherein the transfecting is performed once daily for at least two days and not more than 4 days; and b) expressing the one or more cellular reprogramming factors in the cells resulting in transient reprogramming of the cells, wherein the cells are rejuvenated without dedifferentiation into stem cells.

Embodiment P16. The method of embodiment P15, wherein the one or more cellular reprogramming factors are selected from the group consisting of OCT4, SOX2, KLF4, c-MYC, LIN28 and NANOG.

Embodiment P17. The method of embodiment P16, wherein the cellular reprogramming factors include OCT4, SOX2, KLF4, c-MYC, LIN28 and NANOG.

Embodiment P18. The method of embodiment P15, further including transplanting the rejuvenated cells into the subject.

Embodiment P19. The method of embodiment P15, wherein the age-related disease or condition is a degenerative disease.

Embodiment P20. The method of embodiment P15, wherein the age-related disease or condition is a neurodegenerative disease or a musculoskeletal disorder.

Embodiment P21. A method for treating a subject for a disease or disorder involving cartilage degeneration, the method including: a) transfecting chondrocytes in need of rejuvenation in vivo or ex vivo with one or more non-integrative messenger RNAs encoding one or more cellular reprogramming factors, wherein the transfecting is performed once daily for at least two days and not more than 4 days; and b) expressing the one or more cellular reprogramming factors in the chondrocytes resulting in transient reprogramming of the chondrocytes, wherein the chondrocytes are rejuvenated without dedifferentiation into stem cells.

Embodiment P22. The method of embodiment P21, wherein the one or more cellular reprogramming factors are selected from the group consisting of OCT4, SOX2, KLF4, c-MYC, LIN28 and NANOG.

Embodiment P23. The method of embodiment P22, wherein the cellular reprogramming factors include OCT4, SOX2, KLF4, c-MYC, LIN28 and NANOG.

Embodiment P24. The method of embodiment P21, wherein the disease or disorder involving cartilage degeneration is arthritis.

Embodiment P25. The method of embodiment P24, wherein the arthritis is osteoarthritis or rheumatoid arthritis.

Embodiment P26. The method of embodiment P21, wherein treatment reduces inflammation in the subject.

Embodiment P27. The method of embodiment P21, wherein the transfecting is performed ex vivo and the rejuvenated chondrocytes are transplanted into an arthritic joint of the subject.

Embodiment P28. The method of embodiment P27, wherein the chondrocytes are isolated from a cartilage sample obtained from the subject.

Embodiment P29. The method of embodiment P21, wherein treatment reduces expression of RANKL, iNOS, IL6, IL8, BDNF, IFNα, IFNγ, and LIF and increases expression of SOX9 and COL2A1 by the chondrocytes.

Embodiment P30. The method of embodiment P21, wherein the subject is an elderly subject.

Embodiment P31. The method of embodiment P21, wherein the subject is a mammalian subject.

Embodiment P32. The method of embodiment P31, wherein the mammalian subject is a human subject.

Embodiment P33. A method for treating a disease or disorder involving muscle degeneration in a subject, the method including: a) transfecting skeletal muscle stem cells in vivo or ex vivo with one or more non-integrative messenger RNAs encoding one or more cellular reprogramming factors, wherein the transfecting is performed once daily for at least two days and not more than 4 days; and b) expressing the one or more cellular reprogramming factors in the skeletal muscle stem cells resulting in transient reprogramming of the skeletal muscle stem cells, wherein the skeletal muscle stem cells are rejuvenated without loss of their ability to differentiate into muscle cells.

Embodiment P34. The method of embodiment P33, wherein the one or more cellular reprogramming factors are selected from the group consisting of OCT4, SOX2, KLF4, c-MYC, LIN28 and NANOG.

Embodiment P35. The method of embodiment P34, wherein the cellular reprogramming factors include OCT4, SOX2, KLF4, c-MYC, LIN28 and NANOG.

Embodiment P36. The method of embodiment P33, wherein the transfecting is performed ex vivo and the rejuvenated skeletal muscle stem cells are transplanted into a muscle in need of repair or regeneration in the subject.

Embodiment P37. The method of embodiment P33, wherein the skeletal muscle stem cells are isolated from a muscle sample obtained from the subject.

Embodiment P38. The method of embodiment P33, wherein treatment results in regeneration of myofibers.

Embodiment P39. The method of embodiment P33, wherein treatment restores potency of skeletal muscle stem cells.

Embodiment P40. The method of embodiment P33, wherein the subject is an elderly subject.

Embodiment P41. The method of embodiment P33, wherein the subject is a mammalian subject.

Embodiment P42. The method of embodiment P41, wherein the mammalian subject is a human subject.

### EMBODIMENTS

Embodiment 1. A method of rejuvenating cells, the method including transfecting cells with one or more non-integrative messenger RNAs encoding one or more cellular reprogramming factors for not more than five (5) continuous days, thereby producing rejuvenated cells.

Embodiment 2. The method of embodiment 1, wherein a transcriptomic profile of the rejuvenated cells becomes more similar to a transcriptomic profile of young cells.

Embodiment 3. The method of embodiment 2, wherein the transcriptomic profile of the rejuvenated cells includes an increase in gene expression of one or more genes selected from RPL37, RHOA, SRSF3, EPHB4, ARHGAP18, RPL31, FKBP2, MAP1LC3B2, Elf1, Phf8, Pol2s2, Taf1 and Sin3a.

Embodiment 4. The method of any one of the preceding embodiments, wherein the rejuvenated cells exhibit increased gene expression of one or more nuclear and/or epigenetic markers compared to a reference value.

Embodiment 5. The method of embodiment 4, wherein the marker is selected from HP1gamma, H3K9me3, lamina support protein LAP2alpha, and SIRT1 protein.

Embodiment 6. The method of any one of the preceding embodiments, wherein the rejuvenated cells exhibit increased proteolytic activity compared to a reference value.

Embodiment 7. The method of embodiment 6, wherein increased proteolytic activity is measured as increased cell autophagosome formation, increased chymotrypsin-like proteasome activity, or a combination thereof.

Embodiment 8. The method of any one of the preceding embodiments, wherein the rejuvenated cells exhibit improved mitochondria health and function compared to a reference value.

Embodiment 9. The method of embodiment 8, wherein improved mitochondria health and function is measured as increased mitochondria membrane potential, decreased reactive oxygen species (ROS), or a combination thereof.

Embodiment 10. The method of any one of the preceding embodiments, wherein the rejuvenated cells exhibit decreased expression of one or more SASP cytokines compared to a reference value.

Embodiment 11. The method of embodiment 10, wherein the SASP cytokines include one or more of IL18, IL1A, GROA, IL22, and IL9.

Embodiment 12. The method of any one of the preceding embodiments, wherein the rejuvenated cells exhibit reversal of the methylation landscape.

Embodiment 13. The method of embodiment 12, wherein reversal of the methylation landscape is measured by Horvath clock estimation.

Embodiment 14. The method of any one of embodiments 4-13, wherein the reference value is obtained from an aged cell.

Embodiment 15. The method of any one of the preceding embodiments, wherein transfecting the cells with the messenger RNAs includes a method selected from lipofectamine and LT-1 mediated transfection, dextran-mediated transfection, calcium phosphate precipitation, polybrene mediated transfection, electroporation, encapsulation of the mRNAs in liposomes, and direct microinjection.

Embodiment 16. The method of any one of the preceding embodiments, wherein the one or more cellular reprogramming factors are selected from OCT4, SOX2, KLF4, c-MYC, LIN28 and NANOG.

Embodiment 17. The method of any one of the preceding embodiments, wherein the one or more cellular reprogramming factors include OCT4, SOX2, KLF4, c-MYC, LIN28 and NANOG.

Embodiment 18. The method of any one of the preceding embodiments, wherein the cells are mammalian cells.

Embodiment 19. The method of any one of the preceding embodiments, wherein the cells are human cells.

Embodiment 20. The method of any one of the preceding embodiments, wherein the cells are from an elderly subject.

Embodiment 21. The method of any one of the preceding embodiments, wherein the cells are selected from fibroblasts, endothelial cells, chondrocytes, skeletal muscle stem cells, keratinocytes, mesenchymal stem cells and corneal epithelial cells.

Embodiment 22. The method of embodiment 21 wherein the cells are mesenchymal stem cells.

Embodiment 23. The method of embodiment 22, wherein rejuvenated mesenchymal stem cells exhibit a reduction in senescence parameters (p16, p21 and positive SAβGal staining), increased cell proliferation, and/or a decrease in ROS levels.

Embodiment 24. The method of any one of the preceding embodiments, wherein the method is performed in vitro, ex vivo, or in vivo.

Embodiment 25. The method of embodiment 24 wherein the method is performed in vivo.

Embodiment 26. The method of embodiment 25, wherein the cells are within a tissue or organ.

Embodiment 27. The method of any one of embodiments 25-27, wherein the method reduces numbers of senescent cells within the tissue or organ.

Embodiment 28. The method of any one of embodiments 25-27, wherein the method decreases expression of one or more of IL18, IL1A, GROA, IL22, and IL9.

Embodiment 29. The method of any one of the preceding embodiments, wherein the method restores function, increases potency, enhances viability, increases replicative capacity or life span of the cells, or a combination thereof.

Embodiment 30. The method of any one of embodiments 1-24, wherein the transfecting is performed once daily for 5 days.

Embodiment 31. The method of any one of embodiments 1-24, wherein the transfecting is performed once daily for 4 days.

Embodiment 32. The method of any one of embodiments 1-24, wherein the transfecting is performed once daily for 3 days.

Embodiment 33. The method of any one of embodiments 1-24, wherein the transfecting is performed once daily for 2 days.

Embodiment 34. A method for treating a subject for an age-related disease or condition, a cartilage degeneration disorder, a neurodegenerative disorder, and/or musculoskeletal dysfunction, the method including administering a therapeutically effective amount of cells, wherein the cells include one or more non-integrative messenger RNAs encoding one or more cellular reprogramming factors.

Embodiment 35. The method of embodiment 34, wherein the one or more cellular reprogramming factors are selected from OCT4, SOX2, KLF4, c-MYC, LIN28 and NANOG.

Embodiment 36. The method of any one of embodiments 34-35, wherein the one or more cellular reprogramming factors include OCT4, SOX2, KLF4, c-MYC, LIN28 and NANOG.

Embodiment 37. The method of any one of embodiments 34-36, wherein the subject has an age-related disease or condition.

Embodiment 38. The method of embodiment 34, wherein the age-related disease or condition is selected from an eye, skin, or musculoskeletal dysfunction.

Embodiment 39. The method of any one of embodiments 34-36, wherein the subject has a cartilage degeneration disorder.

Embodiment 40. The method of embodiment 39, wherein the disorder is selected from arthritis, chondrophasia, spondyloarthropathy, ankylosing spondylitis, lupus erythematosus, relapsing polychondritis, and Sjogren's syndrome.

Embodiment 41. The method of any one of embodiments 39 or 40, wherein treating reduces expression of inflammatory factors and/or and increases ATP and collagen metabolism.

Embodiment 42. The method of embodiment 41, wherein the inflammatory factors are selected from RANKL, iNOS2, IL6, IFNα, MCP3 and MIP1A.

Embodiment 43. The method of embodiment 42, wherein ATP and collagen metabolism is measured by one or more of increased ATP levels, decreased ROS and increased SOD2, increased COL2A1 and overall proliferation by the chondrocytes.

Embodiment 44. The method of any one of embodiments 34-36, wherein the subject has a musculoskeletal dysfunction.

Embodiment 45. The method of any one of embodiments 34-44, wherein administering a therapeutically effective amount of cells includes injection or surgical implantation.

Embodiment 46. The method of any one of embodiments 34-45, wherein the therapeutically effective amount of rejuvenated cells is selected from fibroblasts, endothelial cells, chondrocytes, skeletal muscle stem cells, keratinocytes, mesenchymal stem cells and corneal epithelial cells.

Embodiment 47. The method of embodiment 46 wherein the therapeutically effective amount of rejuvenated cells are corneal epithelial cells.

Embodiment 48. The method of embodiment 47, wherein the rejuvenated corneal epithelial exhibit a reduction in senescence parameters.

Embodiment 49. The method of embodiment 48, wherein the senescence parameters include one or more of expression of p21 and p16, mitochondria biogenesis PGC1α, and expression of inflammatory factor IL8.

Embodiment 50. A method for treating a subject for an age-related disease or condition, a cartilage degeneration disorder, and/or subject has a musculoskeletal dysfunction, the method including administering a therapeutically effective amount of one or more non-integrative messenger RNAs encoding one or more cellular reprogramming factors.

Embodiment 51. The method of embodiment 50, wherein the one or more cellular reprogramming factors are selected from OCT4, SOX2, KLF4, c-MYC, LIN28 and NANOG.

Embodiment 52. The method of any one of embodiments 50-51-48, wherein the one or more cellular reprogramming factors include OCT4, SOX2, KLF4, c-MYC, LIN28 and NANOG.

Embodiment 53. The method of any one of embodiments 50-52, wherein the subject has an age-related disease or condition.

Embodiment 54. The method of embodiment 53, wherein the age-related disease or condition is selected from an eye, skin, or musculoskeletal dysfunction.

Embodiment 55. The method of any one of embodiments 50-52, wherein the subject has a cartilage degeneration disorder.

Embodiment 56. The method of embodiment 55, wherein the disorder is selected from arthritis, chondrophasia, spondyloarthropathy, ankylosing spondylitis, lupus erythematosus, relapsing polychondritis, and Sjogren's syndrome.

Embodiment 57. The method of any one of embodiments 50-52, wherein the subject has a subject has a musculoskeletal dysfunction

Embodiment 58. The method of any one of embodiments 50-57, wherein administering a therapeutically effective amount of one or more non-integrative messenger RNAs includes direct injection into a target cell.

Embodiment 59. The method of embodiment 58 wherein the target cell is selected from epithelial cells, endothelial cells, connective tissue cells, muscle cells, and nervous system cells.

Embodiment 60. A method of rejuvenating engineered tissue ex vivo, the method including transfecting the tissue with one or more non-integrative messenger RNAs encoding one or more cellular reprogramming factors for not more than five (5) continuous days, thereby producing rejuvenated engineered tissue.

Embodiment 61. The method of embodiment 60, wherein the engineered tissue exhibits a reduction in senescence parameters, pro-inflammatory factors, improvements in histological score, or a combination thereof.

Embodiment 62. The method of any one of embodiments 60 or 61 wherein the engineered tissue is engineered skin tissue.

Embodiment 63. The method of any one of embodiments 60-62, wherein the senescence parameters are selected from p16 and positive SaβGal staining and pro-inflammatory factors IL8 and MMP1

Embodiment 64. The method of any one of embodiments 60-63, wherein the histological score includes morphology, organization, and/or quality.

Embodiment 65. A pharmaceutical composition including rejuvenated cells, wherein the rejuvenated cells are obtained by transfecting cells with one or more non-integrative messenger RNAs encoding one or more cellular reprogramming factors for not more than five (5) continuous days.

Embodiment 66. The method of any one of the preceding embodiments, wherein the one or more cellular reprogramming factors are selected from OCT4, SOX2, KLF4, c-MYC, LIN28 and NANOG

Embodiment 67. The composition of embodiment 65 or 66, wherein the cells display one or more of the following: increased expression of HP1 gamma, H3K9me3, LAP2alpha, SIRT1, increased mitochondrial membrane potential and decreased reactive oxygen species, and decreased expression of SASP cytokines.

Embodiment 68. The composition of embodiment 67, wherein the SASP cytokines include one or more of IL18, IL1A, GROA, IL22, and IL9.

Embodiment 69. The composition of any one of embodiments 65-68, further including one or more additional components selected from nutrients, cytokines, growth factors, extracellular matrix (ECM) components, antibiotics, anti-oxidants, and immunosuppressive agents.

Embodiment 70. The composition of any one of embodiments 65-69, further including a pharmaceutically acceptable carrier.

Embodiment 71. The composition of any one of embodiments 65-70, wherein the cells are autologous or allogeneic.

1.-19. (canceled)

20. Use of a messenger RNA (mRNA) encoding one or more reprogramming factors selected from the group consisting of Oct4, Sox2, Klf4, cMyc, Lin28, and NANOG in the manufacture of a composition for treating a somatic cell by transfecting the somatic cell with the messenger RNA (mRNA) to express the one or more reprogramming factors in the somatic cell to obtain a rejuvenated cell with retention of cellular identity.

21. The use of claim 20, wherein the composition comprises an excipient for transfection.

22. The use of claim 21, wherein the composition comprises liposomes, wherein the mRNA is encapsulated in the liposomes.

23. The use of claim 20, wherein the composition is for transfecting in vitro, in vivo, or ex vivo.

24. The use of claim 20, wherein said somatic cell is a human cell selected from the group consisting of fibroblasts, endothelial cells, connective tissue cells, chondrocytes, skeletal muscle stem cells, muscle cells, nervous system cells, keratinocytes, mesenchymal stem cells, blood cells, skin cells, immune cells, and corneal epithelial cells.

25. The use of claim 24, wherein said blood cells comprise a macrophage, an erythrocyte, a monocyte, a neutrophil, or an eosinophil, or wherein said immune cells comprise a macrophage, a neutrophil, an eosinophil, a mast cell, a T cell, a B cell, or a natural killer cell.

26. The use of claim 24, wherein said human cell is within a tissue or an organ selected from skin, hair, lung, cartilage, or eye.

27. The use of claim 20, wherein the composition is for transfecting the somatic cell with the mRNA for not more than 5 consecutive days.

28. Use of an mRNA encoding one or more reprogramming factors selected from the group consisting of Oct4, Sox2, Klf4, cMyc, Lin28, and NANOG in the manufacture of a kit for treating an age-related disease or condition in a subject, the kit comprising a composition for transfecting differentiated cells associated with the age-related disease or condition with the mRNA to express the one or more reprogramming factors in the differentiated cells to obtain rejuvenated cells with retention of cellular differentiation.

29. The use of claim 28, wherein the age-related disease or condition is a dermatologic disease or condition, an eye disease or condition, a respiratory disease or condition, or a musculoskeletal disease or condition.

30. The use of claim 29, wherein the dermatologic disease or condition is dermal atrophy, dermal elastolysis, skin wrinkling, sebaceous gland hyperplasia, sebaceous gland hypoplasia, senile lentigo, a pigmentation abnormality, graying hair, hair loss, hair thinning, or a chronic skin ulcer, wherein the eye disease or condition is age-related macular degeneration, glaucoma, a cataract, dry eye, diabetic retinopathy, or vision loss, wherein the respiratory disease or condition is pulmonary fibrosis, chronic obstructive pulmonary disease, asthma, chronic bronchitis, pulmonary embolism, lung cancer or a lung infection, and wherein the musculoskeletal disease or condition is arthritis, osteoporosis, myeloma, gout, Paget's disease, bone fracture, bone marrow failure syndrome, ankyloses, diffuse idiopathic skeletal hyperostosis, hematogenous osteomyelitis, muscle atrophy, peripheral neuropathy, multiple sclerosis, amyotrophic lateral sclerosis, Duchene muscular dystrophy, primary lateral sclerosis, or myasthenia gravis.

31. The use of claim 28, wherein the composition is configured for transfecting the differentiated cells to achieve expression of the one or more reprogramming factors for a period of time sufficient to rejuvenate the differentiated cells while retaining their identity.

32. Use of a messenger RNA (mRNA) encoding one or more reprogramming factors selected from the group consisting of Oct4, Sox2, Klf4, cMyc, Lin28, and NANOG for transfecting a somatic cell to express the one or more reprogramming factors in the somatic cell to obtain a rejuvenated cell with retention of cellular identity.

33. The use of claim 32, wherein the mRNA is encapsulated in liposomes.

34. The use of claim 32, wherein the mRNA is for transfecting in vitro, in vivo, or ex vivo.

35. The use of claim 32, wherein said somatic cell is a human cell selected from the group consisting of fibroblasts, endothelial cells, connective tissue cells, chondrocytes, skeletal muscle stem cells, muscle cells, nervous system cells, keratinocytes, mesenchymal stem cells, blood cells, skin cells, immune cells, and corneal epithelial cells.

36. The use of claim 35, wherein said blood cells comprise a macrophage, an erythrocyte, a monocyte, a neutrophil, or an eosinophil, or wherein said immune cells comprise a macrophage, a neutrophil, an eosinophil, a mast cell, a T cell, a B cell, or a natural killer cell.

37. The use of claim 35, wherein said human cell is within a tissue or an organ selected from skin, hair, lung, cartilage, or eye.

38. The use of claim 32, wherein the mRNA is for transfecting the somatic cell for not more than 5 consecutive days.
